(19) United States
(12) Patent Application Publication
(10) Pub. No.: US 2025/0009679 A1
(43) Pub. Date: Jan. 9, 2025

(54) CATIONIC LIPID-BASED NANOCARRIER AND PREPARATION METHOD AND APPLICATION THEREOF

(71) Applicant: National Defense Medical Center, Taipei City (TW)

(72) Inventors: Jenn-Jong Young, Taipei City (TW); Cheng-Cheung Chen, Taipei City (TW); Chuan-Chang Chuang, Taipei City (TW); Yu-Hsiu Chang, Taipei City (TW); Hui-Ju Yen, Taipei City (TW); Xin-An Chen, Taipei City (TW)

(21) Appl. No.: 18/528,408

(22) Filed: Dec. 4, 2023

(30) Foreign Application Priority Data
Jul. 7, 2023 (TW) 112125447

(51) Int. Cl. A61K 9/51; A61K 9/127; A61K 45/06

(52) U.S. Cl. CPC A61K 9/5192; A61K 9/1271; A61K 9/5123; A61K 9/5146; A61K 9/5161; A61K 45/06

(57) ABSTRACT

A method for preparing a cationic lipid-based nanocarrier includes providing an organic solvent and an aqueous solution, adding compositions for forming a lipid-based nanocarrier and at least one kind of cationic polysaccharides respectively into the organic solvent and the aqueous solution according to respective solubility thereof, and flowing the organic solvent and the aqueous solution through a microfluidic device under the control of a micromixer, so as to mix the organic solvent and the aqueous solution in the microfluidic device, thereby obtaining the cationic lipid-based nanocarrier in a single-step process.

### CROSS-REFERENCE TO RELATED APPLICATION

This application claims priority to Taiwan Patent Application No. 112125447, filed on Jul. 7, 2023. The entire contents of the above-mentioned patent application are incorporated herein by reference for all purposes.

### FIELD OF THE INVENTION

The present disclosure relates to a cationic lipid-based nano-carrier (LNC), and a preparation method and applications thereof, and more particularly to a cationic LNC which is coated with polyelectrolytes via hydrogen bonds, ionic bonds and/or covalent bonds, a preparation method where the cationic LNCs with bioactive or functional substances embedded therein are formed in a single-step process, and the applications thereof.

### BACKGROUND OF THE INVENTION

Many drugs or bioactive substances must be transported by carriers to specific cells or tissues. LNCs are often used as delivery system for therapeutic or diagnostic substances, such as small molecule drugs, fluorescent molecules, photosensitizers, nanometals, quantum dots, peptides, proteins (antibodies) and/or nucleic acids etc. The most commonly used LNC is the liposome (LPS). LPS can be used for delivering poorly soluble hydrophobic drugs, or for delivering lipophilic and hydrophilic drugs simultaneously. Furthermore, LPS is biocompatible, biodegradable and non-toxic. Not only the efficacy and stability but also the safety of embedded drugs is enhanced. Besides various LPS-carrying drugs on the market, another common type of LNC is the lipid nanoparticle (LNP). The preparation of LNP is similar to that of LPS, but ionizable lipid or cationic lipid is added for forming of micelles embedded with nucleic acid and for succeeding assembly with helper lipids to form LNP. Therefore, LNP is different from LPS in morphology. Under TEM (transmission electron microscope), LNP has a uniform and solid core while LPS is hollow or multi-layered.

The conventional preparation process of LNC is as followed. First, all compositions are dissolved in an organic solvent (usually chloroform or dichloromethane). Then, the organic solvent is removed through evaporation and a thin film on the glass surface is formed. An aqueous solution is added to the thin film to produce LNC through phase inversion, followed by ultrasonication to disperse the solution, and extrusion to squeeze the larger or multi-lamellar bilayer particle into a small uni-lamellar structure. Then, LNC is precipitated and extracted by ultracentrifugation. In addition, if polymer-coated LNCs are desired, the obtained LNCs are mixed with polymer solution and stirred over a period of time. The entire process is time-consuming, the chlorine-containing organic solvents pollute the environment, and a large amount of remaining is generated during extrusion, resulting in low yield and high production cost. Moreover, large particle sizes and high polydispersity resulting from unstandardized phase inversion make LNCs ineffective delivery systems. Besides, equipment for extrusion and ultracentrifugation during mass production further increases the cost. In addition, parameter setting needs to be tailored according to batch size and it poses a scale-up challenge.

The compositions of LNC are the key factor that affects its delivery. It is mainly composed of phospholipids, along with optional addition of sterols, polyethylene glycol (PEG) derivatives of phospholipids, and/or surfactants. Phospholipid can be natural, such as soy-PC or egg-PC, or synthetic. While natural phospholipid is cost-efficient, synthetic phospholipid comes in better purity which results in more stable LNC. The basic structure of phospholipid is a phosphatidic acid (PA)-type phospholipid which includes one glycerol molecule connected to two saturated or unsaturated fatty acids with C16-C22 chain, and one phosphate molecule. PA-type phospholipid can be further connected with different polar molecules to form different types of phospholipids, for example, phosphatidylcholine (PC) type, phosphatidylethanolamine (PE) type, phosphatidylserine (PS) type, phosphatidylinositol (PI) type, and phosphatidylglycerol (PG) type. Among them, PC- and PE-type phospholipids are zwitterionic and therefore electrically neutral, while PS-, PI-, PG- and PA-type phospholipids are negatively charged; thus, most of the LNCs prepared by these phospholipids are neutral or negatively-charged on the surface. In addition, to increase the circulation and residence time of LNC in the blood, some phospholipids therein are replaced by polyethylene glycol (PEG) derivatives of PE. The most used sterol is cholesterol, and an increase in its molar ratio increases the rigidity and the radius of LNC. Surfactants increase the embedding rate of insoluble bioactive or functional substances and are administrated according to practical conditions.

Improvement of therapeutic effect is achieved by a carrier, so that bioactive or functional substances are released to desired location, and side effects caused by off-target tissue distribution are reduced, taking for example anticancer drugs, vaccines or immunogenic drugs, nucleic acids, and some antibacterial drugs are often desired at specific tissue and sometimes intracellularly. If a ligand with high binding selectivity to the target cells is added to the carrier, with receptor-mediated endocytosis (RME) effect, endocytosis of the carrier by target cells is increased, thereby reducing the side effect, and improving the treatment efficiency. Furthermore, due to its amphiphilicity, PEG is easily exposed on the outer surface of LNC which provides an ideal location for the ligand with target-binding function. Therefore, when PEG is bonded with small molecular compound, protein (antibody) or aptamer, identification of specific cells and selective targeting delivery of LNC can be further improved.

Size and uniformity of LNC affect circulation, residence time, and tissue infiltration of the carrying drug, which are key factors influencing the efficacy of the LNC-carried drug. However, due to inability to accurately control the microenvironment in conventional preparation of LNC, particle size and the polydispersity of LNC are controlled by means of ultrasonication and the extrusion process, in which various steps are involved and standardization is compromised, thereby leading to lower encapsulation rates and lower loading capacities rates of the drug.

Microfluidic technology makes a great improvement in preparation of LNC. It accurately controls the microenvironment to produce particles in continuous flow mode. Many designs of microfluidic structures are available in the market. The advantage of adapting microfluidic technology is that the particle sizes of the prepared LNCs are precisely modulated by the computer-controlled process parameters, and that uniform LNCs can be produced rapidly with high reproducibility. More importantly, the optimized process parameters for one single microfluidic device can be directly transferred to multiple microfluidic devices with identical geometries without complex and time-consuming adaptation, which makes the scaling-up of process easier. Therefore, microfluidic technology can not only effectively control the particle size and uniformity of LNC, but also make the conversion from laboratory scale to factory scale smoother.

Therefore, there is a need to provide an improved cationic lipid-based nano-carrier and a preparation method thereof for improving the defects in the prior arts.

### SUMMARY OF THE INVENTION

An object of the present disclosure is to provide a method for preparing a cationic LNC via a single-step process in which formation of LNC and coating of cationic polysaccharides on the outer surface of LNC via different bonds, such as, hydrogen bonds, ionic bonds, covalent bonds or a mixture thereof, are achieved simultaneously.

Another object of the present disclosure is to provide a cationic LNC with great endocytic uptake efficiency by various cells and the ability of target drug delivery. Furthermore, the cationic LNC is also capable of successfully and massively delivering nucleic acids and proteins into cells (nuclei) and rapidly increasing antibody titers after vaccination.

In accordance with an aspect of the present disclosure, a method for preparing a cationic LNC is provided. The method includes providing an organic solvent and an aqueous solution, adding compositions for forming a LNC and at least one kind of cationic polysaccharides respectively into the organic solvent and the aqueous solution according to respective solubility thereof, and flowing the organic solvent and the aqueous solution through a microfluidic device under the control of a micromixer so as to mix the organic solvent and the aqueous solution in the microfluidic device, thereby obtaining the positively surface-charged LNC in a single-step process.

In an embodiment, the cationic LNC is formed by coating the at least one kind of cationic polysaccharides on an outer surface of the LNC via hydrogen bonds, ionic bonds, covalent bonds or a combination thereof.

In an embodiment, the at least one kind of cationic polysaccharides includes at least one selected from the group consisting of N-[(2-hydroxy-3-trimethylammonium)propyl]chitosan chloride (HTCC) and N-trimethylchitosan (TMC), and the at least one kind of cationic polysaccharides has a molecular weight of 5-1000 kDa, a degree of deacetylation (DD) of 50-99%, and a degree of quaternization (DQ) of 20-90%.

In an embodiment, a flow rate ratio (FRR) of the aqueous solution and the organic solvent is controlled by the micromixer at 10:1 to 1:10, a total flow rate (TFR) is controlled by the micromixer at 1 to 40 mL/min, and a total lipid concentration (TLC) is of 1 to 100 mg/mL.

In an embodiment, the compositions for forming the LNC include phospholipid and at least one selected from the group consisting of cholesterol, polyethylene glycol (PEG) derivative of phospholipid, and ionizable lipid or cationic lipid.

In an embodiment, the phospholipid is phosphatidic acid (PA) type phospholipid including one glycerol molecule connected to two saturated or unsaturated fatty acids with C16-C22 chain and one phosphate molecule.

In an embodiment, the PA-type phospholipid is further connected with a polar molecule to form at least one type of phospholipid selected from the group consisting of phosphatidylcholine (PC) type, phosphatidylethanolamine (PE) type, phosphatidylserine (PS) type, phosphatidylinositol (PI) type, and phosphatidylglycerol (PG) type.

In an embodiment, the polyethylene glycol (PEG) derivative of phospholipid includes a phosphatidylethanolamine (PE) type phospholipid connected with a polyethylene glycol having a molecular weight of 500-1000000 Da.

In an embodiment, one end of the polyethylene glycol (PEG) derivative of phospholipid is connected with one selected from the group consisting of folic acid, biotin, mannose, galactose, and cRGD peptide, or is connected with a conjugating molecule selected from the group consisting of N-Hydroxysuccinimide (NHS), N-maleimide, orthopyridyl disulfide, and vinylsulfone.

In an embodiment, the ionizable lipid or cationic lipid includes a C16-C100 saturated or unsaturated fatty acid chain with a primary amine, a secondary amine, a tertiary amine or a quaternary amine.

In an embodiment, further including a step of adding a bioactive or functional substance to be delivered by the cationic LNC into the organic solvent or the aqueous solution according to the solubility thereof.

In an embodiment, the bioactive or functional substance includes at least one selected from the group consisting of small molecular drug, peptide, protein, nucleic acid, fluorescent molecule, photosensitive reagent, nanometal and quantum dots.

In an embodiment, the organic solvent and the aqueous solution have dissolved therein 10-100 mole % of soy-PC, egg-PC, PC-type phospholipid, PE-type phospholipid, PS-type phospholipid, PI-type phospholipid, PG-type phospholipid, PA-type phospholipid or a combination thereof, 0-50 mole % of cholesterol, 0-50 mole % of PEG derivative of PE-type phospholipid, 0-20 mole % of bioactive or functional substances, and 0.1-40 mg/mL of HTCC, TMC or a combination thereof, and wherein the organic solvent comprises one selected from the group consisting of alcohol with less than or equal to 4 carbons, Dimethyl sulfoxide (DMSO), Dimethylformamide (DMF) or a mixed solution thereof, and the aqueous solution comprises water or a buffer solution.

In an embodiment, the organic solvent and the aqueous solution have dissolved therein 10-100 mole % of soy-PC, egg-PC, PC-type phospholipid, PE-type phospholipid, PS-type phospholipid, PI-type phospholipid, PG-type phospholipid, PA-type phospholipid or a combination thereof, 0-50 mole % of cholesterol, 0-50 mole % of PEG derivative of PE-type phospholipid, 1-60 mole % of ionizable lipid or cationic lipid, 1-20 mole % of nucleic acid, and 0.1-40 mg/mL of HTCC, TMC or a combination thereof, and wherein the organic solvent comprises one selected from the group consisting of alcohol with less than or equal to 4 carbons, Dimethyl sulfoxide (DMSO), Dimethylformamide (DMF) or a mixed solution thereof, and the aqueous solution comprises water or a buffer solution.

In an embodiment, the cationic LNC has a particle size ranged between 10 nm and 500 nm and a zeta potential ranged between +2 mV and +60 mV.

In an embodiment, the LNC includes at least one of a liposome and a lipid nanoparticle.

In accordance with another aspect of the present disclosure, a cationic LNC prepared by the method described above is provided.

In an embodiment, the cationic LNC is used as one selected from the group consisting of a protein carrier, a nucleic acid carrier, a small molecular drug carrier and a vaccine adjuvant.

### BRIEF DESCRIPTION OF THE DRAWINGS

The above contents of the present disclosure will become more readily apparent to those ordinarily skilled in the art after reviewing the following detailed description and accompanying drawings, in which:

FIG. 1 shows influences of total flow rate (TFR) and flow rate ratio (FRR) on particle size of liposome;

FIG. 2 shows the uptakes by J774A.1 macrophage cells of liposomes respectively connecting with ligands of folic acid (fa4LPS-fitc), biotin (bi4LPS-fitc), mannose (ma4LPS-fitc) and galactose (ga4LPS-fitc) at 5 μg/mL and 20 μg/mL;

FIG. 3A shows the uptakes by J774A.1 macrophage cells of liposome without polysaccharides coated (meLPS-fitc), liposomes respectively coated with negatively charged chondroitin sulfate (meLPS-c10-fitc), fucoidan (meLPS-f10-fitc) and alginic acid (meLPS-a10-fitc), and liposomes respectively coated with HTCC (meLPS-h10-fitc) and TMC (meLPS-t10-fitc), where each liposome is cultured with J774A.1 macrophage cells for 16 hours at 20 μg/mL;

FIG. 3B shows the uptakes by the J774A.1 macrophage cells of liposomes coated with HTCC at different concentrations, where each group of liposome is cultured with J774A.1 macrophage cells for 16 hours;

FIG. 4 is a schematic view illustrating the cationic liposome coated with HTCC or TMC via hydrogen bonds, ionic bonds and covalent bonds;

FIG. 5 shows TEM images of representative liposomes prepared by a single-step microfluidic process listed in Table 1;

FIGS. 6A-6C show the uptakes by JAWS II dendritic cells of liposome without coating and liposomes coated with HTCC at different concentrations via hydrogen bonds, as detected by flow cytometry, wherein culture time is 16 hours;

FIGS. 7A-7C show the uptakes by JAWS II dendritic cells of liposome without coating and liposomes coated with HTCC at different concentrations via covalent bonds, as detected by flow cytometry, wherein culture time is 16 hours;

FIGS. 8A-8C show the uptakes by JAWS II dendritic cells of liposome without coating and liposomes coated with TMC at different concentrations via hydrogen bonds, as detected by flow cytometry, wherein culture time is 16 hours;

FIGS. 9A-9C show the uptakes by JAWS II dendritic cells of liposome without coating and liposomes coated with TMC at different concentrations via covalent bonds, as detected by flow cytometry, wherein culture time is 16 hours;

FIG. 10A shows the uptakes of liposomes coated with TMC via hydrogen bonds at 4 μg/mL respectively by cancer cells (KB cells, NCI-H460 cells, OE cells, H1299 cells and HeLa cells) and by L929 cells, as detected by flow cytometry, and the quantitative relationships between that by each kind of cancer cells and L929 cells, wherein culture time is 16 hours;

FIG. 10B shows the uptakes of liposomes coated with HTCC via hydrogen bonds at 4 μg/mL respectively by cancer cells (KB cells, NCI-H460 cells, OE cells, H1299 cells and HeLa cells) and by L929 cells, as detected by flow cytometry, and the quantitative relationships between that by each kind of cancer cells and L929 cells, wherein culture time is 16 hours;

FIG. 11A shows the uptakes of liposomes coated with TMC via covalent bonds at 4 μg/mL respectively by cancer cells (KB cells, NCI-H460 cells, OE cells, H1299 cells and HeLa cells) and by L929 cells, as detected by flow cytometry, and the quantitative relationships between that each kind of cancer cells and L929 cells, wherein culture time is 16 hours;

FIG. 11B shows the uptakes of liposomes coated with HTCC via covalent bonds at 4 μg/mL respectively by cancer cells (KB cells, NCI-H460 cells, OE cells, H1299 cells and HeLa cells) and by L929 cells, as detected by flow cytometry, and the quantitative relationships between that by each kind of cancer cells and L929 cells, wherein culture time is 16 hours;

FIG. 12A shows influences of concentrations of liposomes and coating concentrations of HTCC via hydrogen bonds respectively on cell viability of J774A.1 cells, wherein culture time is 24 hours;

FIG. 12B shows results of cytotoxicity assays of liposomes at different concentrations and liposomes coated with HTCC of different concentrations via hydrogen bonds respectively co-cultured with J774A.1 cells, wherein culture time is 24 hours;

FIG. 13A shows influences of liposomes coated with HTCC or TMC via hydrogen bonds, ionic bonds and covalent bonds respectively on cell viability of JAWS II cells, wherein culture time is 24 hours;

FIG. 13B shows results of cytotoxicity assays of liposomes coated with HTCC or TMC via hydrogen bonds, ionic bonds and covalent bonds respectively co-cultured with JAWS II cells, wherein culture time is 24 hours;

FIG. 14A shows results of cytotoxicity assays of liposomes coated with HTCC at different concentrations via covalent bonds towards JAWS II cells, wherein culture time is 24 hours;

FIG. 14B shows results of cytotoxicity assays of mixtures of AVA and liposomes at different concentrations which are respectively coated with HTCC via hydrogen bonds or covalent bonds towards JAWS II cells, wherein culture time is 24 hours;

FIG. 15 shows results of apoptosis assays of liposomes which are respectively coated with HTCC or TMCC via hydrogen bonds or covalent bonds, as detected by flow cytometry, wherein liposomes at 20 μg/mL are cultured with JAWS II cells for 16 hours, and results are compared with that of liposome without coating HTCC or TMC and control group;

FIG. 16 shows cytotoxicity assays of liposomes respectively coated with HTCC and TMC via hydrogen bonds at different concentrations cultured with L929 cells and cancer cells (KB cells, OE cells, HeLa cells, H1299 cells and NCI-H460 cells) at 37° C. for 24 hours;

FIG. 17 shows cytotoxicity assays of liposomes respectively coated with HTCC and TMC via covalent bonds at different concentrations cultured with L929 cells and cancer cells (KB cells, OE cells, HeLa cells, H1299 cells and NCI-H460 cells) at 37° C. for 24 hours;

FIG. 18A shows cytokine levels (IFN-γ, IL-10, TNF-α and IL-12) derived from co-culture of JAWS II cells and liposomes which are respectively coated with HTCC and TMC via hydrogen bonds at 37° C. for 24 hours, wherein concentration of liposomes is 5 μg/mL;

FIG. 18B shows cytokine levels (IFN-γ, IL-10, TNF-α and IL-12) derived from co-culture of JAWS II cells and liposomes which are respectively coated with HTCC and TMC via covalent bonds at 37° C. for 24 hours, wherein concentration of liposomes is 5 μg/mL;

FIG. 19A shows protective antigen (PA)-specific IgG responses in BALB/c mice at two-weeks post-priming and two-weeks post-boosting in group meLPS-h4, scLPS-h4, AVA, AVA+meLPS-h4 and AVA+scLPS-h4, wherein *p<0.05, **p<0.01, and ***p<0.001;

FIG. 19B shows IgG1 and IgG2a subclass distributions in BALB/c mice at two-weeks post-boosting in group AVA, AVA+meLPS-h4 and AVA+scLPS-h4, wherein *p<0.05, **p<0.01, and ***p<0.001;

FIG. 20A shows protective antigen (PA)-specific IgG responses in BALB/c mice at two-weeks post-priming and two-weeks post-boosting in group meLPS-t4, scLPS-t4, AVA, AVA+meLPS-t4 and AVA+scLPS-t4, wherein *p<0.05, **p<0.01, and ***p<0.001;

FIG. 20B shows IgG1 and IgG2a subclass distribution in BALB/c mice at two-weeks post-boosting in group AVA, AVA+meLPS-t4 and AVA+scLPS-t4, wherein *p<0.05, **p<0.01, and ***p<0.001;

FIG. 21A shows fluorescent images of 293T cells after 48 hours of co-culture with EGFP DNA plasmids that are mixed with meLPS-t10 or meLPS-h10 which are respectively coated with TMC and HTCC via hydrogen bonds in a ratio of 1:20;

FIG. 21B shows fluorescent images of 293T cells after 48 hours of co-culture with EGFP plasmids that are mixed with scLPS-t10 or scLPS-h10 which are respectively coated with TMC and HTCC via covalent bonds, pgLPS-h10 which is coated with HTCC via ionic bonds, and pgLPS-h10+scLPS-h10 which is respectively coated with TMC and HTCC via covalent bonds and ionic bonds in a ratio of 1:20;

FIG. 22 shows fluoresces generated from EGFP mRNAs which are delivered into JAWS II cells by different kinds of cationic liposomes in a RNA-to-LPS ratio of 1:20;

FIG. 23A shows adsorptions of DNA to the cationic liposomes in different ratios;

FIG. 23B shows fluorescent images of 293T cells after 48 hours of co-culture with EGFP plasmid DNAs which are mixed with different cationic LPSs in ratios of 1:2 and 1:4;

FIG. 24 shows fluorescent images of 293T cells after co-culture with Cy5-CTP-labeled EGFP mRNA under two kinds of fluorescence after mixing with scLPS-h10 in a ratio of 1:4;

FIG. 25 shows fluorescent images of 293T cells after 48 hours of co-culture with the neutral LNP with DNA embedded therein (LNP-DNA) and the cationic LNP with DNA embedded therein (LNP-HTCC-DNA), which are respectively prepared in the single microfluidic step at different concentrations; and

FIG. 26 shows fluorescent images of various cells cultured with the neutral LNP with DNA embedded therein (LNP-DNA) and the cationic LNP with DNA embedded therein (LNP-HTCC-DNA).

### DETAILED DESCRIPTION OF THE PREFERRED EMBODIMENT

The present disclosure will now be described more specifically with reference to the following embodiments. It is to be noted that the following descriptions of preferred embodiments of this disclosure are presented herein for purpose of illustration and description only. It is not intended to be exhaustive or to be limited to the precise form disclosed.

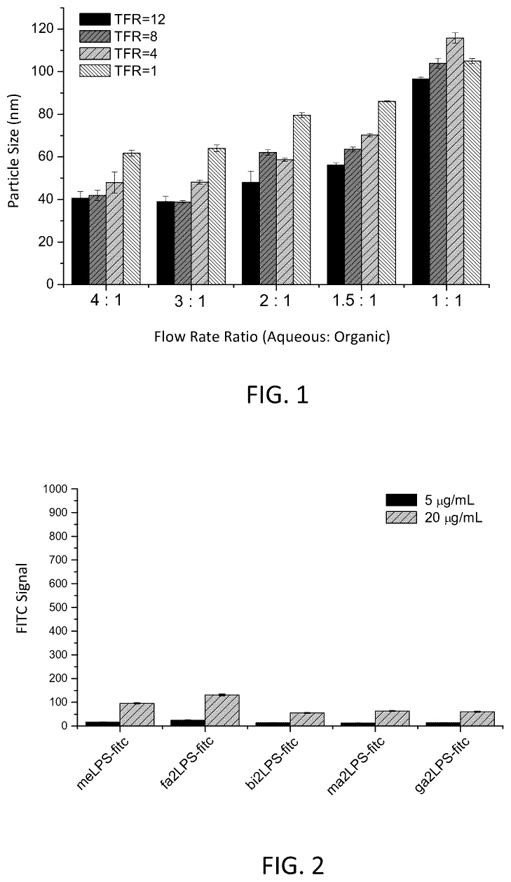

Please refer to FIG. 1, which shows influences of total flow rate (TFR) and flow rate ratio (FRR) on particle size of liposome (LPS). Organic phase is ethanol, aqueous phase is PBS (pH 7.4), compositions in organic phase are soy PC, cholesterol and DSPE-PEG5K-X in a molar ratio of 48:42:10, and total lipid concentration (TLC) in organic phase is 10 mg/mL. TFR and FRR are controlled by a micromixer. As shown in FIG. 1, at FRRs of aqueous to organic phase of 4:1, 3:1, 2:1, 1.5:1 and 1:1, and at TFRs of respectively 1, 4, 8 and 12 mL/min, the particle sizes of LPS are precisely between 40 nm and 120 nm. To wit, controller parameters, e.g., TFR and FRR, of the micromixer, grant particle sizes of LPS from 40 to 120 nm with high reproducibility. Customized flow rate of micromixer can go up to tens of milliliters per minute, where concentration can reach 100 mg/mL. That is, at a FRR of aqueous to organic phase of 10:1 to 1:10, a TFR of 1 to 40 mL/min, and a TLC of 1 to 100 mg/mL, particle sizes of LPS are correspondingly dependent on the compositions.

Generally, during LNC preparation, to achieve high selectivity for cell surface receptors and consequent increased endocytosis via RME effect, ligands, such as folic acid, biotin, mannose, galactose, or cRGD peptide-conjugated target molecule are connected onto LNC surface at PEG ends by replacing the PEG derivatives in the phospholipids. Here, identical process parameters can be remained since this replacement does not change the molar ratio significantly.

Please refer to FIG. 2 which shows the uptakes by J774A.1 macrophages of LPS connecting respectively with ligands of folic acid (fa4LPS-fitc), biotin (bi4LPS-fitc), mannose (ma4LPS-fitc) and galactose (ga4LPS-fitc) on PEG at 5 μg/mL and 20 μg/mL. In the present disclosure, cellular uptake is assessed under flow cytometry, where fluorescent materials are embedded into LPSs through adding 1 mg/mL of fluorescein isothiocyanate (FITC) to the organic phase as prepared. As shown in FIG. 2, even with ligands identifying specific cell surface receptors connected to the outer surface of LPS, the uptake efficiency of LPS by cells is poor.

Experiments disclose that uptake efficiency and ratio of LPS by cells are increased by coating the outer surface of LPS with cationic polysaccharides, whereby the efficacy of LPS as a delivery system can be improved. The cationic polysaccharides include N-[(2-hydroxy-3-trimethylammonium)propyl]chitosan chloride (HTCC) and N-trimethylchitosan (TMC). Details are disclosed below.

The compositions of LPSs prepared by the microfluidic method of the present disclosure are listed in Table 1.

TABLE 1

| | | | | | Aqueous |
|---|---|---|---|---|---|
| | | | | | Phase |
| | | | | | PBS |
| | Organic Phase | | | | (pH 7.4) |
| LPS | Molar ratio (%) | | | mg/mL | PEs |
| Abbreviation | Lipids | Cholesterol | DSPE-PEG5K-X | FITC | (mg/mL) |
| meLPS(-fitc) | Soy PC 48 | 42 | 10 (X = methyl) | 0 (1) | — |
| fa4LPS(-fitc) | Soy PC 48 | 42 | 6 (X = Me), | 0 (1) | — |
| | | | 4 (X = folic acid) | | |
| bi4LPS(-fitc) | Soy PC 48 | 42 | 6 (X = Me), | 0 (1) | — |
| | | | 4 (X = biotin) | | |
| ma4LPS(-fitc) | Soy PC 48 | 42 | 6 (X = Me), | 0 (1) | — |
| | | | 4 (X = mannose) | | |
| ga4LPS(-fitc) | Soy PC 48 | 42 | 6 (X = Me), | 0 (1) | — |
| | | | 4 (X = galactose) | | |
| amLPS(-fitc) | Soy PC 48 | 42 | 10 (X = NH₂) | 0 (1) | — |
| meLPS-t10(-fitc) | Soy PC 48 | 42 | 10 (X = methyl) | 0 (1) | TMC (10) |
| meLPS-h10(-fitc) | Soy PC 48 | 42 | 10 (X = methyl) | 0 (1) | HTCC (10) |
| meLPS-c10(-fitc) | Soy PC 48 | 42 | 10 (X = methyl) | 0 (1) | ChS (10) |
| meLPS-f10(-fitc) | Soy PC 48 | 42 | 10 (X = methyl) | 0 (1) | FUC (10) |
| meLPS-a10(-fitc) | Soy PC 48 | 42 | 10 (X = methyl) | 0 (1) | ALG (10) |
| meLPS-t8(-fitc) | Soy PC 48 | 42 | 10 (X = methyl) | 0 (1) | TMC (8) |
| meLPS-t6(-fitc) | Soy PC 48 | 42 | 10 (X = methyl) | 0 (1) | TMC (6) |
| meLPS-t4(-fitc) | Soy PC 48 | 42 | 10 (X = methyl) | 0 (1) | TMC (4) |
| meLPS-t2(-fitc) | Soy PC 48 | 42 | 10 (X = methyl) | 0 (1) | TMC (2) |
| meLPS-h8(-fitc) | Soy PC 48 | 42 | 10 (X = methyl) | 0 (1) | HTCC (8) |
| meLPS-h6(-fitc) | Soy PC 48 | 42 | 10 (X = methyl) | 0 (1) | HTCC (6) |
| meLPS-h4(-fitc) | Soy PC 48 | 42 | 10 (X = methyl) | 0 (1) | HTCC (4) |
| meLPS-h2(-fitc) | Soy PC 48 | 42 | 10 (X = methyl) | 0 (1) | HTCC (2) |
| scLPS(-fitc) | Soy PC 48 | 42 | 10 (X = SCM) | 0 (1) | — |
| scLPS-h4(-fitc) | Soy PC 48 | 42 | 10 (X = SCM) | 0 (1) | HTCC (4) |
| scLPS-t4(-fitc) | Soy PC 48 | 42 | 10 (X = SCM) | 0 (1) | TMC (4) |
| scLPS-h10(-fitc) | Soy PC 48 | 42 | 10 (X = SCM) | 0 (1) | HTCC (10) |
| scLPS-t10(-fitc) | Soy PC 48 | 42 | 10 (X = SCM) | 0 (1) | TMC (10) |
| pgLPS | Soy PC 28 | 42 | 10 (X = methyl) | 0 | — |
| | DSPG 20 | | | | |
| pgLPS-t10 | Soy PC 28 | 42 | 10 (X = methyl) | 0 | TMC (10) |
| | DSPG 20 | | | | |
| pgLPS-h10 | Soy PC 28 | 42 | 10 (X = methyl) | 0 | HTCC (10) |
| | DSPG 20 | | | | |

Composition = Soy PC:Cholesterol:DSPE-PEG5K-X (48:42:10 mol %); TLC = 10 mg/mL; FRR = 3:1 (Aqu./Org.); TFR = 8 mL/min; Organic phase: ethanol; Aqueous phase: PBS (pH 7.4); and FITC = 1 mg/mL in ethanol. The group of scLPS-h4(-fitc), scLPS-t4(-fitc), scLPS-h10(-fitc) and scLPS-t10(-fitc) represents covalent combinations. The group of pgLPS-t10 and pgLPS-h10 represents ionic combinations.

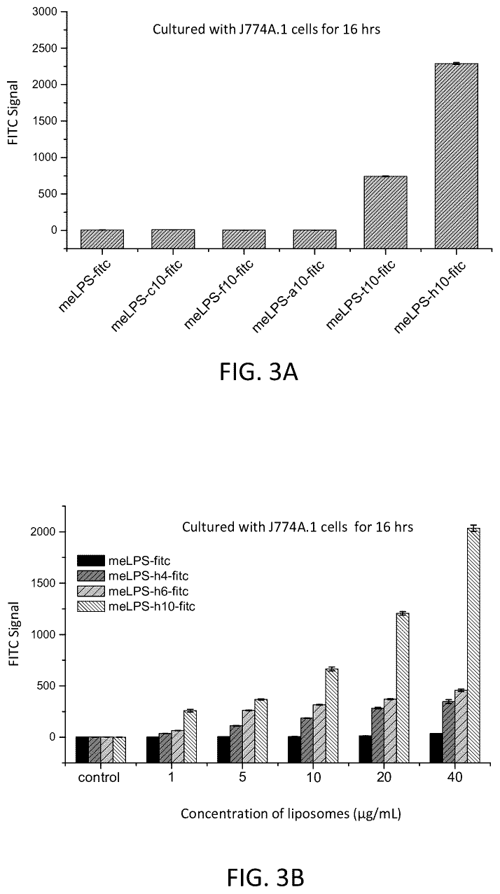

Please refer to FIG. 3A which shows the uptakes by J774A.1 macrophages of, respectively, LPS without polysaccharides coated (meLPS-fitc), LPSs coated with negatively charged chondroitin sulfate (meLPS-c10-fitc), fucoidan (meLPS-f10-fitc) and alginic acid (meLPS-a10-fitc), and LPSs coated with HTCC (meLPS-h10-fitc) or TMC (meLPS-t10-fitc), where each group of LPS is cultured with J774A.1 macrophages for 16 hours at 20 μg/mL. As shown in FIG. 3A, compared with LPS without polysaccharides coated and LPSs coated with negatively charged polysaccharides, the uptake efficiency and ratio of LPSs coated with cationic polysaccharides, namely coated with HTCC (meLPS-h10-fitc) or TMC (meLPS-t10-fitc), are significantly higher. No difference is seen between the uptake efficiency and ratio of LPSs coated with negatively charged polysaccharides and that of LPS without polysaccharides coated.

Please refer to FIG. 3B which shows the uptakes by J774A.1 macrophage cells of LPSs coated with HTCC at different concentrations, respectively, where each group of LPS is cultured with J774A.1 macrophages for 16 hours. As shown in FIG. 3B, the higher the concentration of coated HTCC on LPS, the more the uptake of LPS by J774A.1 macrophages. Here, meLPS-h4-fitc represents 4 mg/mL of HTCC; meLPS-h6-fitc represents 6 mg/mL of HTCC; and meLPS-h10-fitc represents 10 mg/mL of HTCC in LPS formulation.

Accordingly, cellular uptake is increased by coated cationic polysaccharides on the surface of LPSs, which means cationic-LPS-based carrier provides better therapeutic efficiency.

The following discussion is about how cellular uptake efficiency of cationic LNC is increased. Positively charged polysaccharides are coated on the surface of LNC via hydrogen bonds or van der Waals forces. Enhancement of the bonding force improves stability and performance of LNC as a carrier or adjuvant. Experiments disclose that, during phase inversion, specific functional groups (or negative charges) in phospholipid or PEG-Lipid of LNC bond with hydroxyl groups (—OH), amine groups (—NH2) or cations in polysaccharides in different ways, for example, via hydrogen bonds, ionic bonds, covalent bonds or a combination. Stability and performance of the cationic LNC are therefore improved.

Moreover, with microfluidic technology, LNC preparation and coating of cationic polysaccharides via different bonds (e.g., covalent bonds, ionic bonds, hydrogen bonds or a combination thereof) are complete in a single step. Conventionally, coating of polyelectrolytes or other polymers comes after LNC synthesis, which also takes multiple steps. Obtained LNCs are mixed with polyelectrolytes or polymers over a period to complete the formation of coated LNCs. The whole process is complicated. On the contrary, in the present disclosure, both LNC formation and coating of cationic polysaccharides are completed in the microfluidic device simultaneously. Compositions of LNC, cationic polysaccharides, and the bioactive or functional substances to be embedded are dissolved respectively in organic or aqueous solution according to their solubility. Both solutions flow through the microfluidic device under the control of a micromixer at, for example, a FRR of aqueous to organic phase of 10:1 to 1:10, a TFR of 1 to 40 mL/min, and a TLC of 1 to 100 mg/mL. Cationic LNCs coated with polysaccharides are hence formed in a single step, and the cationic LNC has a particle size ranged between 10 nm and 500 nm and a zeta potential ranged between +2 mV and +60 mV. In other words, preparation of cationic LNC of the present disclosure is condensed where only one single step is needed for formation of LNC and coating of cationic polysaccharides. Not only the preparation process is significantly simplified, but the production yield is also greatly increased.

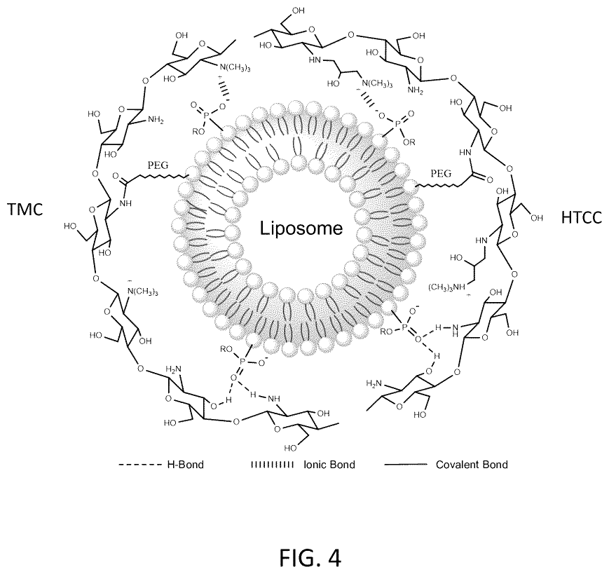

Please refer to FIG. 4 which is a schematic view illustrating the cationic LPS coated with HTCC or TMC via hydrogen bonds, ionic bonds and covalent bonds. Compared with hydrogen bonds or van der Waals forces, ionic bonds and covalent bonds are much stronger, and the cationic LPS formed with the latter two is thereby more stable with higher zeta potential. The following description describes methods for connecting polysaccharides to the outer surface of LPS via ionic bonds and covalent bonds. The first method is to replace all or a portion of phospholipids in the composition with negatively charged ones, such as PS-, PI-, PG-, or PA-type phospholipids, for stronger negative surface charge on LPS which favors ionic bond formation with positively charged HTCC or TMC. The second method is to extend the PEG end of PEG-lipid in the compositions with an NHS (N-hydroxysuccinimide) activated carboxyl group for forming amide bonds or ester bonds with amino groups or hydroxyl groups in HTCC or TMC, which results in a covalent bond with the polysaccharides. Since the compositions described above do not vary greatly, parameters for the single-step microfluidic preparation require no amendment.

In a preferred embodiment, ingredients for the single-step microfluidic preparation of LPS include 10-100 mole % of soy-PC, egg-PC, PC-type phospholipid, PE-type phospholipid, PS-type phospholipid, PI-type phospholipid, PG-type phospholipid, PA-type phospholipid or a combination thereof, 0-50 mole % of cholesterol, 0-50 mole % of PEG derivative of PE-type phospholipid, 0-20 mole % of bioactive or functional substances (such as small molecular drugs, peptides, proteins, nucleic acids, fluorescent molecules, photosensitive reagents, nanometals and quantum dots, etc.), and 0.1-40 mg/mL of HTCC, TMC or a combination thereof. All ingredients are dissolved in organic or aqueous solution according to their respective solubility and injected into microfluidic device simultaneously, from which LPS coated with cationic polysaccharides is obtained in one single step. Here, the organic solvent refers to alcohols with less than or equal to 4 carbons, DMSO (Dimethyl sulfoxide), DMF (Dimethylformamide) or a mixed solution thereof, and the aqueous solution refers to water or various buffer solutions.

In another preferred embodiment, ingredients for the single-step microfluidic preparation of LNP include 10-100 mole % of soy-PC, egg-PC, PC-type phospholipid, PE-type phospholipid, PS-type phospholipid, PI-type phospholipid, PG-type phospholipid, PA-type phospholipid or a combination thereof, 0-50 mole % of cholesterol, 0-50 mole % of PEG derivative of PE-type phospholipid, 1-60 mole % of ionizable lipid or cationic lipid, 1-20 mole % of nucleic acid, and 0.1-40 mg/mL of HTCC, TMC or a combination thereof. All ingredients are dissolved in organic or aqueous solution according to their respective solubility and injected into the microfluidic device simultaneously, from which LNP coated with cationic polysaccharides is obtained in one single step. Here, the organic solvent refers to alcohols with less than or equal to 4 carbons, DMSO (Dimethyl sulfoxide), DMF (Dimethylformamide) or a mixed solution thereof, and the aqueous solution refers to water or various buffer solutions.

In PEG derivatives of PE-type phospholipid, the molecular weight of PEG ranges from 500 to 1000000 Da. The end of a PEG derivative of PE-type phospholipid may be connected to a target molecule that is recognized by a special cell surface receptor, for example, folic acid, biotin, mannose, galactose, or cRGD peptide. Alternatively, the end of a PEG derivative of PE phospholipid may carry a conjugating molecule that binds with peptides, proteins (antibodies) or aptamers, for example, N-maleimide, orthopyridyl disulfide, and vinylsulfone etc.

The ionizable lipid or cationic lipid is composed of a C16-C100 saturated or unsaturated fatty acid chain with a primary amine, a secondary amine, a tertiary amine or a quaternary amine.

Preferably, the cationic polysaccharides, HTCC and TMC, has a molecular weight of 5-1000 kDa, a degree of deacetylation (DD) of 50-99%, and a degree of quaternization (DQ) of 20-90%.

Depending on different types of phospholipids used, the bonding between the cationic polysaccharides and LNC varies accordingly. For example, when the phospholipids are of PC-type and/or PE-type, hydrogen bonds are formed between LNC and cationic polysaccharides; when the phospholipids are of PS-type, PI-type, PG-type and/or PA-type, ionic or hydrogen bonds are formed between LNC and cationic polysaccharides; when the end of a PEG derivative of phospholipid is connected to NHS activated carboxyl group, covalent or hydrogen bonds are formed between LNC and cationic polysaccharides; and when the end of a PEG derivative of phospholipid is connected to a target molecule, which is recognized by a special cell surface receptor, for example, folic acid, biotin, mannose, galactose, or cRGD peptide, any kind of bonds can be formed between LNC and cationic polysaccharides, such as covalent, ionic, hydrogen bonds, or a combination thereof.

The particle size, the zeta potential (ζ) and the polydispersity index (PDI) of LPSs prepared from compositions listed in Table 1 through single-step microfluidic process are listed in Table 2.

TABLE 2

| LPS | As-prepared | | | Dialyzed | | |
|---|---|---|---|---|---|---|
| Abbreviation | Size (nm) | ζ (mV) | PDI | Size (nm) | ζ (mV) | PDI |
| meLPS | 38.8 ± 0.6 | −1.0 ± 1.1 | 0.17 ± 0.02 | 43.3 ± 0.5 | −0.9 ± 2.2 | 0.24 ± 0.01 |
| fa4LPS | 40.9 ± 0.4 | −3.4 ± 0.3 | 0.15 ± 0.02 | 43.8 ± 0.8 | −1.9 ± 0.2 | 0.24 ± 0.06 |
| bi4LPS | 43.4 ± 1.1 | −1.9 ± 0.2 | 0.21 ± 0.03 | 48.8 ± 6.2 | −1.3 ± 0.4 | 0.23 ± 0.02 |
| ma4LPS | 44.8 ± 0.8 | −2.0 ± 0.7 | 0.24 ± 0.05 | 44.7 ± 1.6 | −1.8 ± 0.3 | 0.23 ± 0.03 |
| ga4LPS | 41.3 ± 0.5 | −2.6 ± 0.2 | 0.21 ± 0.02 | 42.2 ± 0.5 | −1.6 ± 0.1 | 0.23 ± 0.03 |
| meLPS-t10 | 53.2 ± 0.9 | 14.7 ± 4.4 | 0.18 ± 0.02 | 53.8 ± 0.4 | 7.6 ± 1.7 | 0.09 ± 0.01 |
| meLPS-h10 | 60.1 ± 0.6 | 17.2 ± 4.7 | 0.21 ± 0.01 | 57.1 ± 3.0 | 7.2 ± 2.5 | 0.19 ± 0.03 |
| meLPS-c10 | 57.7 ± 2.5 | −9.3 ± 1.1 | 0.21 ± 0.06 | 61.5 ± 1.8 | −16.8 ± 5.8 | 0.26 ± 0.06 |
| meLPS-f10 | 56.0 ± 0.3 | −5.5 ± 2.1 | 0.16 ± 0.04 | 60.3 ± 4.3 | −3.9 ± 3.3 | 0.24 ± 0.06 |
| meLPS-a10 | 62.9 ± 1.9 | −16.2 ± 0.2 | 0.21 ± 0.01 | 61.3 ± 2.9 | −9.9 ± 0.8 | 0.20 ± 0.03 |
| meLPS-t8 | 52.3 ± 0.8 | 16.5 ± 5.3 | 0.18 ± 0.02 | 53.8 ± 0.4 | 6.1 ± 1.9 | 0.09 ± 0.01 |
| meLPS-t6 | 55.4 ± 4.7 | 10.8 ± 2.6 | 0.18 ± 0.03 | 55.9 ± 4.0 | 6.6 ± 2.8 | 0.13 ± 0.08 |
| meLPS-t4 | 51.5 ± 5.3 | 7.3 ± 2.5 | 0.19 ± 0.06 | 55.1 ± 3.3 | 6.0 ± 2.5 | 0.18 ± 0.03 |
| meLPS-t2 | 50.3 ± 0.9 | 3.9 ± 2.8 | 0.15 ± 0.06 | 55.0 ± 0.9 | 2.9 ± 1.4 | 0.12 ± 0.03 |
| meLPS-h8 | 58.5 ± 0.9 | 16.5 ± 5.3 | 0.19 ± 0.01 | 56.0 ± 1.0 | 6.1 ± 1.9 | 0.20 ± 0.11 |
| meLPS-h6 | 57.0 ± 0.5 | 10.8 ± 2.6 | 0.18 ± 0.03 | 54.9 ± 1.4 | 6.6 ± 2.8 | 0.17 ± 0.08 |
| meLPS-h4 | 58.6 ± 0.9 | 9.2 ± 1.9 | 0.20 ± 0.02 | 56.0 ± 1.2 | 5.9 ± 1.3 | 0.15 ± 0.09 |
| meLPS-h2 | 57.1 ± 1.0 | 8.1 ± 1.3 | 0.18 ± 0.01 | 55.4 ± 1.7 | 6.2 ± 2.2 | 0.19 ± 0.11 |
| scLPS | 87.4 ± 1.8 | −5.6 ± 0.1 | 0.57 ± 0.09 | 81.5 ± 2.0 | −5.2 ± 0.4 | 0.59 ± 0.12 |
| scLPS-t4 | 107.7 ± 0.5 | 9.8 ± 0.5 | 0.45 ± 0.00 | 95.2 ± 2.3 | 6.7 ± 0.4 | 0.46 ± 0.04 |
| scLPS-t10 | 157.9 ± 3.1 | 14.0 ± 1.0 | 0.42 ± 0.05 | 97.4 ± 2.5 | 9.3 ± 0.8 | 0.52 ± 0.09 |
| scLPS-h4 | 120.4 ± 2.3 | 13.6 ± 0.6 | 0.61 ± 0.06 | 114.3 ± 1.6 | 13.0 ± 0.6 | 0.48 ± 0.01 |
| scLPS-h10 | 158.2 ± 3.9 | 17.4 ± 1.6 | 0.36 ± 0.02 | 95.2 ± 2.3 | 17.7 ± 1.0 | 0.53 ± 0.09 |
| pgLPS | 105.1 ± 0.4 | −1.8 ± 0.5 | 0.18 ± 0.00 | 101.5 ± 1.6 | −2.5 ± 0.6 | 0.19 ± 0.01 |
| pgLPS-t10 | 126.6 ± 0.4 | 26.3 ± 1.7 | 0.26 ± 0.00 | 95.3 ± 0.5 | 12.8 ± 1.1 | 0.25 ± 0.01 |
| pgLPS-h10 | 117.7 ± 0.6 | 21.4 ± 1.3 | 0.25 ± 0.01 | 107.0 ± 0.9 | 20.9 ± 0.9 | 0.26 ± 0.01 |

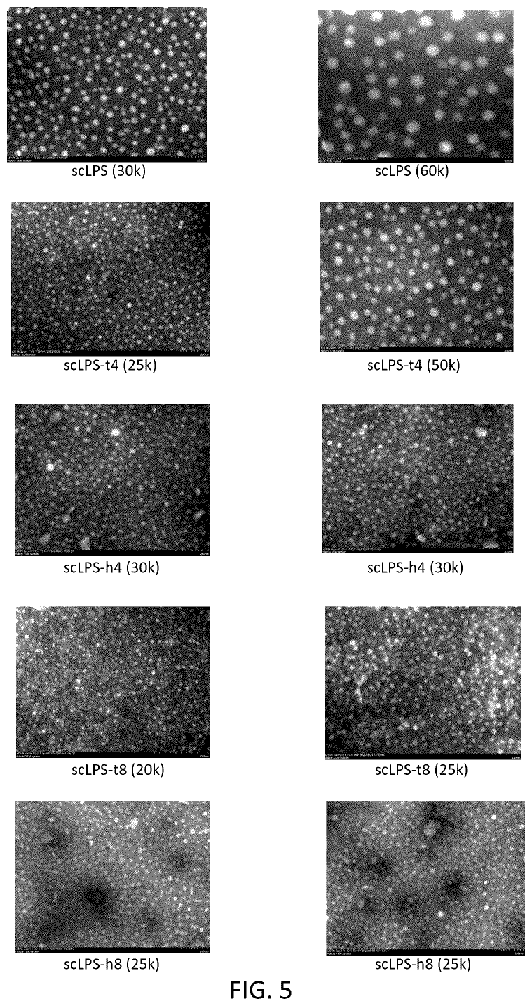

According to the standard errors and PDIs listed in Table 2, high reproducibility of LPSs prepared by the single-step microfluidic process of the present disclosure is seen. According to TEM images shown in FIG. 5, the distribution of particle sizes of cationic LPSs prepared in the same batch is uniform. Generally, the particle size of LPS coated by cationic polysaccharides is slightly larger than that of LPS without coating polysaccharides and ranges between 40-160 nm. The absolute value of zeta potential of cationic-polysaccharides-coated LPS is also greater than that of LPS without coating polysaccharides and ranges between 0-30 mV, where the value thereof is positively correlated with the concentration of polysaccharides. The prepared LPS is placed in a dialysis cassette and submerged in PBS, of about 300 times the volume of LPS solution, until the organic solvent is purged completely—for subsequent in vitro or in vivo application.

### Influence of Cationic LPS on Uptake by Immune Cells

No significant difference is seen in the uptake rate by J774A.1 macrophages between LPS carrying introduced ligand, which can be recognized by the special cell surface receptor, and LPS without coating polysaccharides due to the zeta potential which is close to zero or negative. Meanwhile, prominent increase in the uptake rate by J774A.1 macrophages is seen in LPS coated with positively charged polysaccharides via hydrogen bonds (as shown in FIG. 3A and FIG. 3B). This phenomenon is also observed in JAWS II dendritic cells.

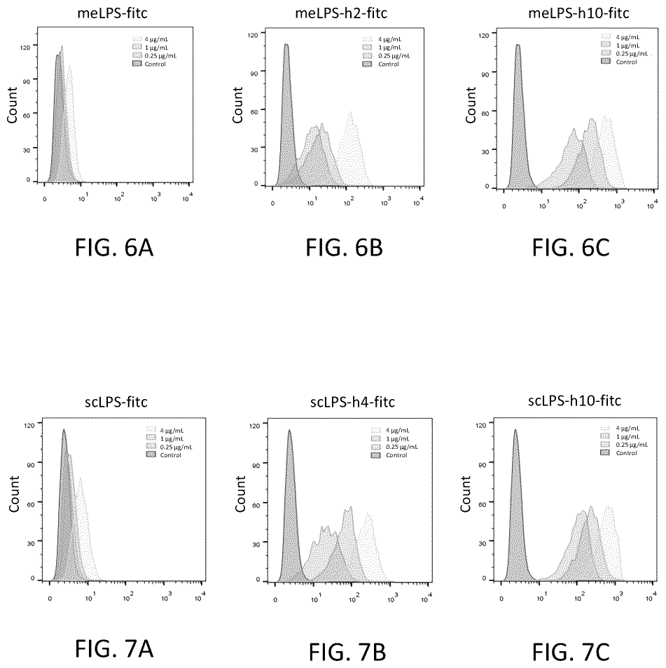

Please refer to FIGS. 6A-6C which show the uptakes (Median fluorescence intensity) by JAWS II dendritic cells of LPS without coating (Control) and LPSs coated with HTCC at different concentrations (meLPS-fitc, meLPS-h2-fitc and meLPS-h10-fitc) via hydrogen bonds, as detected by flow cytometry, wherein culture time is 16 hours. Detailed data is listed in Table 3.

TABLE 3

| Concentration | | MFI (Median fluorescence intensity) | | |
|---|---|---|---|---|
| of LPS | | meLPS-fitc | meLPS-h2-fitc | meLPS-h10-fitc |
| Control | | 2.07 ± 0.04 | 2.07 ± 0.04 | 2.07 ± 0.04 |
| 0.25 | μg/mL | 3.32 ± 0.02 | 11.43 ± 0.21 | 64.54 ± 2.81 |
| 1 | μg/mL | 2.65 ± 0.02 | 18.90 ± 0.36 | 186.69 ± 2.31 |
| 4 | μg/mL | 4.43 ± 0.04 | 118.33 ± 3.06 | 482.67 ± 9.07 |

Please refer to FIGS. 7A-7C which show the uptakes (Median fluorescence intensity) by JAWS II dendritic cells of LPS without coating (Control) and LPSs coated with HTCC at different concentrations (scLPS-fitc, scLPS-h4-fitc and scLPS-h10-fitc) via covalent bonds, as detected by flow cytometry, wherein culture time is 16 hours. Detailed data is listed in Table 4.

TABLE 4

| Concentration | | MFI (Median fluorescence intensity) | | |
|---|---|---|---|---|
| of LPS | | scLPS-fitc | scLPS-h4-fitc | scLPS-h10-fitc |
| Control | | 2.09 ± 0.03 | 2.09 ± 0.03 | 2.09 ± 0.03 |
| 0.25 | μg/mL | 2.58 ± 0.02 | 20.16 ± 0.51 | 107.67 ± 2.08 |
| 1 | μg/mL | 3.14 ± 0.04 | 65.37 ± 1.69 | 213.33 ± 4.04 |
| 4 | μg/mL | 6.26 ± 0.05 | 224.66 ± 4.16 | 586.34 ± 4.15 |

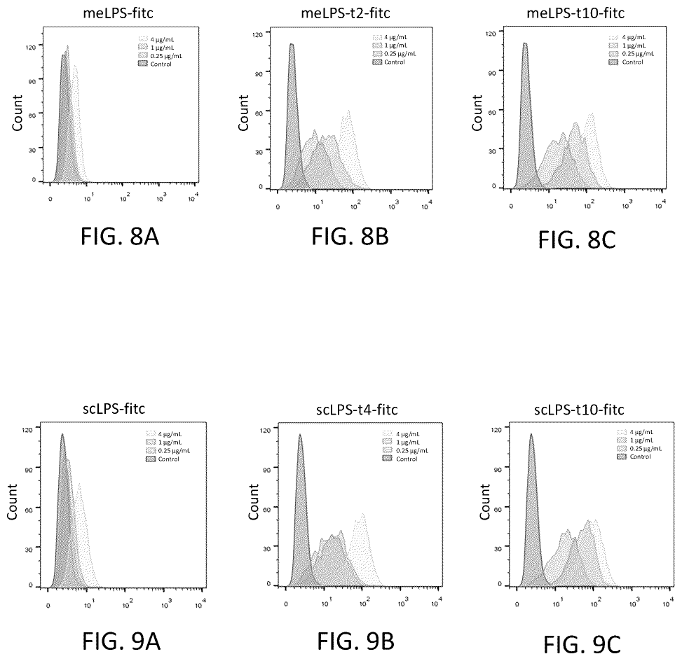

Please refer to FIGS. 8A-8C which show the uptakes (Median fluorescence intensity) by JAWS II dendritic cells of LPS without coating (Control) and LPSs coated with TMC at different concentrations (meLPS-fitc, meLPS-t2-fitc and meLPS-t10-fitc) via hydrogen bonds, as detected by flow cytometry, wherein culture time is 16 hours. Detailed data is listed in Table 5.

TABLE 5

| Concentration | | MFI (Median fluorescence intensity) | | |
|---|---|---|---|---|
| of LPS | | meLPS-fitc | meLPS-t2-fitc | meLPS-t10-fitc |
| Control | | 2.07 ± 0.04 | 2.07 ± 0.04 | 2.07 ± 0.04 |
| 0.25 | μg/mL | 3.32 ± 0.02 | 9.10 ± 0.12 | 17.14 ± 0.42 |
| 1 | μg/mL | 2.65 ± 0.02 | 19.23 ± 0.51 | 46.39 ± 1.02 |
| 4 | μg/mL | 4.43 ± 0.04 | 65.80 ± 1.91 | 110.66 ± 2.08 |

Please refer to FIGS. 9A-9C which show the uptakes (Median fluorescence intensity) by JAWS II dendritic cells of LPS without coating (Control) and LPSs coated with TMC at different concentrations (scLPS-fitc, scLPS-t4-fitc and scLPS-t10-fitc) via covalent bonds, as detected by flow cytometry, wherein culture time is 16 hours. Detailed data is listed in Table 6.

TABLE 6

| | |
|---|---|
| Concentration | MFI (Median fluorescence intensity) |

| of LPS | scLPS-fitc | scLPS-t4-fitc | scLPS-t10-fitc |
|---|---|---|---|
| Control | 2.09 ± 0.03 | 2.09 ± 0.03 | 2.09 ± 0.03 |

| | | | | |
|---|---|---|---|---|
| 0.25 | μg/mL | 2.58 ± 0.02 | 13.67 ± 0.31 | 17.74 ± 0.23 |
| 1 | μg/mL | 3.14 ± 0.04 | 17.93 ± 0.50 | 51.87 ± 0.45 |
| 4 | μg/mL | 6.26 ± 0.05 | 82.71 ± 0.66 | 84.96 ± 1.22 |

As shown, no matter whether HTCC is coated via hydrogen bonds or covalent bonds, the uptake (Median fluorescence intensity) of the fluorescent LPS by JAWS II dendritic cells is positively correlated with the concentration of LPS and the concentration of coated HTCC (as shown in FIGS. 6A-6C and FIGS. 7A-7C). Similar results are also observed in LPSs coated with TMC via hydrogen bonds or covalent bonds (as shown in FIGS. 8A-8C and FIGS. 9A-9C). It is deduced that the zeta potential on the surface of LPS is the main factor that determines the cellular uptake. In general, the cell surface is negatively charged, and positively-charged LPSs are attracted to them due to electrostatic attraction, and thus, the uptake rate is increased with the increase of positive value of zeta potential.

Targeting Delivery Ability to Cancer Cells of Cationic LPSs Coated with HTCC or TMC

Given that the uptake rates of the cationic LPS coated with HTCC or TMC by two kinds of immune cells (J774A.1 and JAWS II) are both increased, the difference in uptake between cancer cells and normal cells is further discussed. Experiments are performed on three common cancer cell lines, including cervical cancer cells (KB cells and HeLa cells), lung cancer cells (NCI-H460 cells and H1299 cells) and esophageal cancer cells (OE cells).

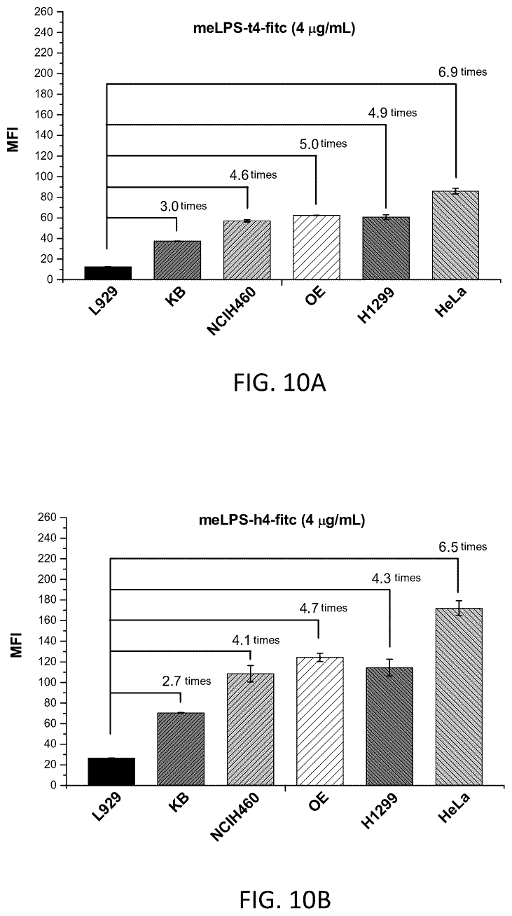

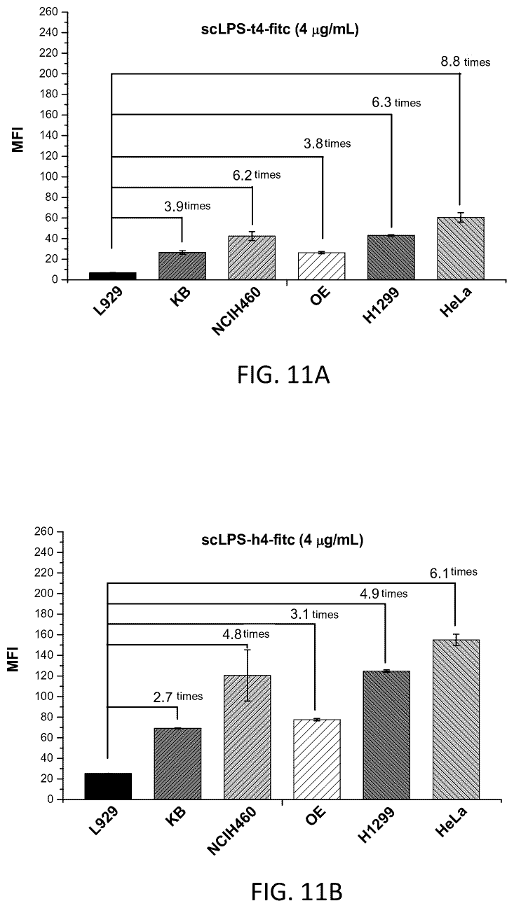

Please refer to FIGS. 10A-10B and FIGS. 11A-11B. FIG. 10A and FIG. 10B respectively indicate the uptakes (MFI) of LPSs coated with TMC and HTCC via hydrogen bonds at 4 μg/mL by cancer cells (KB cells, NCI-H460 cells, OE cells, H1299 cells and HeLa cells) and by L929 cells, as detected by flow cytometry, and the quantitative relationships between that by each kind of cancer cells and that by L929 cells, wherein culture time is 16 hours. FIG. 11A and FIG. 11B respectively indicate the uptakes (MFI) of LPSs coated with TMC and HTCC via covalent bonds at 4 μg/mL by cancer cells (KB cells, NCI-H460 cells, OE cells, H1299 cells and HeLa cells) and by L929 cells, as detected by the flow cytometry, and the quantitative relationships between that by each kind of cancer cells and that by L929 cells, wherein culture time is 16 hours.

According to the results shown in FIGS. 10A-10B and FIGS. 11A-11B, no matter whether LPS is coated with HTCC or TMC via hydrogen bonds or covalent bonds, the uptake rates by different kinds of cancer cells are 2.7-8.8 times that of normal cells, which means the selectivity to cancer cells of the HTCC- or TMC-coated LPS is higher than that of normal cells. In theory, the difference is caused by higher negative charge density on the surface of cancer cells than on normal cells, which leads to greater electrostatic attraction and increased uptake rate. Cell surface receptors potentially plays a role, too. There may be specific receptors on cancer cells which bind with chitosan and/or its quaternary derivatives with high selectivity, and RME effect enhances uptake of cationic LPS coated with HTCC or TMC by cancer cells.

Moreover, at the same concentration, uptake of HTCC-coated LPS by cancer cells is greater than that of TMC-coated LPS. This phenomenon is mainly due to the difference of DQ (degree of quaternization) of HTCC and TMC. % DQ is calculated from ¹H NMR data according to equation (1):

$$\% DQ = \{ [ {( {CH}_{3} )}_{3} ] / [ {COCH}_{3} ] \times 1 / 3 \times ( 1 - \% DD ) \} \qquad ( 1 )$$

wherein % DD represents degree of deacetylation, [(CH3)₃] is integral of chemical shift of trimethylammonium at 3.3 ppm, and [COCH₃] is integral of single peak at 2.4 ppm.

% DQ calculated according to equation (1) respectively for HTCC and TMC is 69.8% and 41%. A high DQ value indicates polysaccharides with a higher percentage of positively charged quaternized sugar residues, and accordingly, HTCC-coated LPS with greater zeta potential is internalized by cancer cells more easily. It once again proves that the level of positive zeta potential is the most critical factor for the uptake rate of LPS by cells.

### Cytotoxicity Assay of Cationic LPS

Although the level of positive zeta potential is the most critical factor for the uptake rate of LPS by cells, the positively charged LPS is usually cytotoxic to some extent to different cells. Cytotoxicity of cationic LPS is further analyzed.

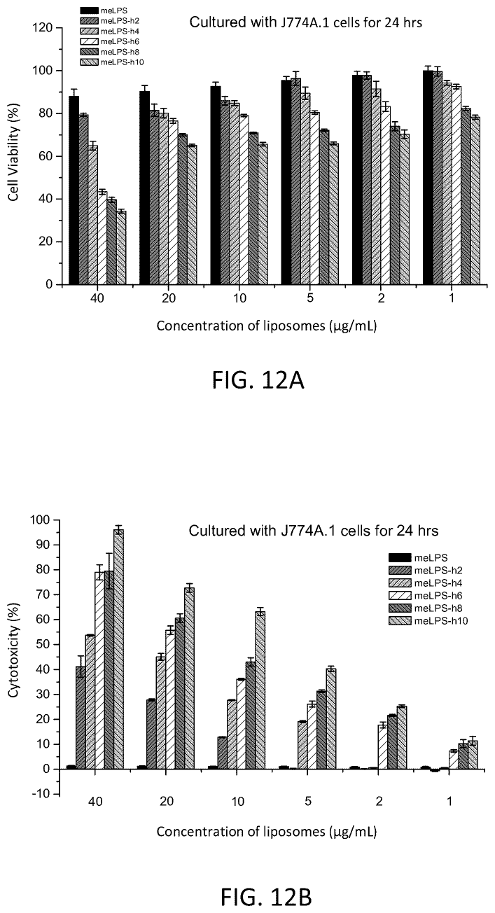

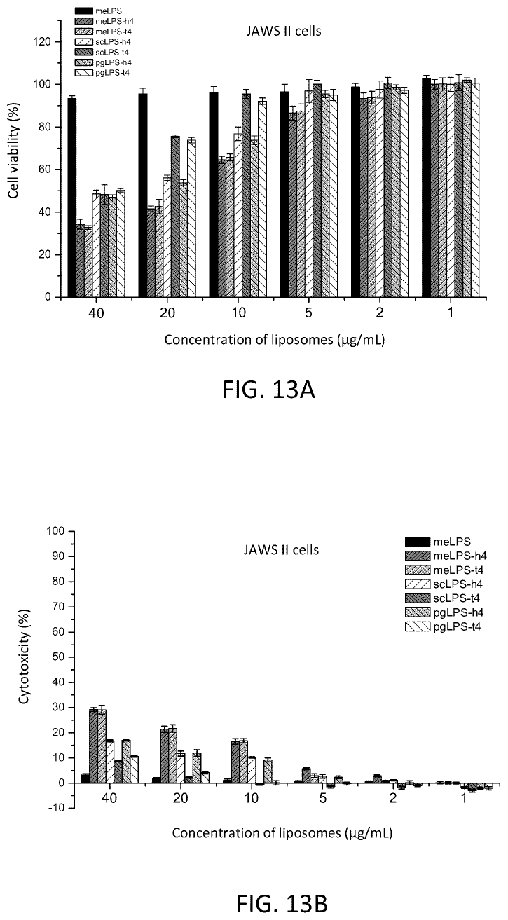

Please refer to FIG. 12A, FIG. 12B, FIG. 13A and FIG. 13B. FIG. 12A shows influences of concentrations of LPSs and coating concentrations of HTCC via hydrogen bonds respectively on cell viability of J774A.1 cells, wherein culture time is 24 hours. FIG. 12B shows results of cytotoxicity assays (LDH assays) of LPSs at different concentrations and LPSs coated with HTCC of different concentrations via hydrogen bonds respectively co-cultured with J774A.1 cells, wherein culture time is 24 hours. FIG. 13A shows influences of LPSs coated with HTCC or TMC via hydrogen bonds, ionic bonds and covalent bonds respectively on cell viability of JAWS II cells, wherein culture time is 24 hours. FIG. 13B shows results of cytotoxicity assays (LDH assays) of LPSs coated with HTCC or TMC via hydrogen bonds, ionic bonds and covalent bonds respectively co-cultured with JAWS II cells, wherein culture time is 24 hours. Cell viability is assessed by MTT assay and results are shown in percentage (%). Cytotoxicity is assessed by LDH assay and results are shown in percentage (%).

According to results shown in FIG. 12A and FIG. 12B, compared with LPS without coating (meLPS and scLPS), the cationic LPSs show some toxicity to J774A.1 cells, and the toxicity increases as concentrations of LPS and coated HTCC increase. Similar results can be found in JAWS II cells co-culture (not shown), and are not repeated here. However, according to results shown in FIG. 13A and FIG. 13B, compared with LPSs coated with HTCC via hydrogen bonds (meLPS-h4), the ones coated with HTCC via ionic bonds (pgLPS-h4) or via covalent bonds (scLPS-h4) exhibit lower toxicity to JAWS II dendritic cells at the same concentration, whereas the cell viability is higher. Similar results can be found regarding TMC-coated LPSs.

As most LPSs are not cytotoxic, toxicity of cationic LPS majorly comes from the coated cationic polysaccharides. When a cationic polysaccharide forms a hydrogen bond with LPS, it easily dissociates due to the weaker bonding strength of hydrogen bonds, and the cytotoxicity of the formulation is similar to that of the cationic polysaccharide itself. However, when LPS is coated via covalent bonds or ionic bonds, dissociation of cationic polysaccharide is restrained due to the stronger bonding force, and the cytotoxicity thereof is reduced, leading to the results shown in FIG. 13A and FIG. 13B.

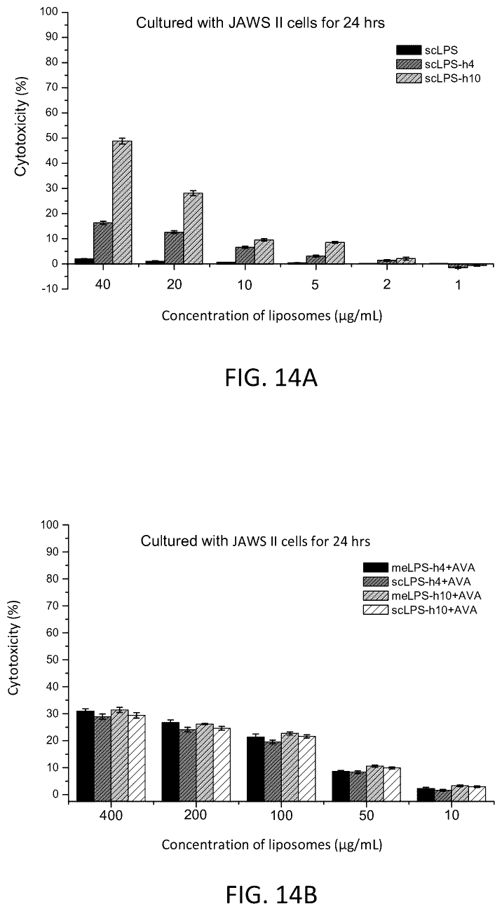

Cytotoxicity of a mixture of the cationic LPS with AVA (a subunit recombinant vaccine) is further tested for investigating its applications in adjuvant formulation and vaccine delivery. Please refer to FIG. 14A and FIG. 14B. FIG. 14A shows results of cytotoxicity assays (LDH assays) of LPSs coated with HTCC at different concentrations via covalent bonds towards JAWS II cells, wherein culture time is 24 hours. FIG. 14B shows results of cytotoxicity assays (LDH assays) of mixtures of AVA and LPSs at different concentrations, respectively coated with HTCC via hydrogen bonds or covalent bonds, towards JAWS II cells, wherein culture time is 24 hours.

As shown, cytotoxicity of cationic LPS is greatly reduced after mixing with AVA, to as low as one tenth. It is presumed that negative charge of the recombinant protein neutralizes the positive zeta potential of LPS and reduces its cytotoxicity.

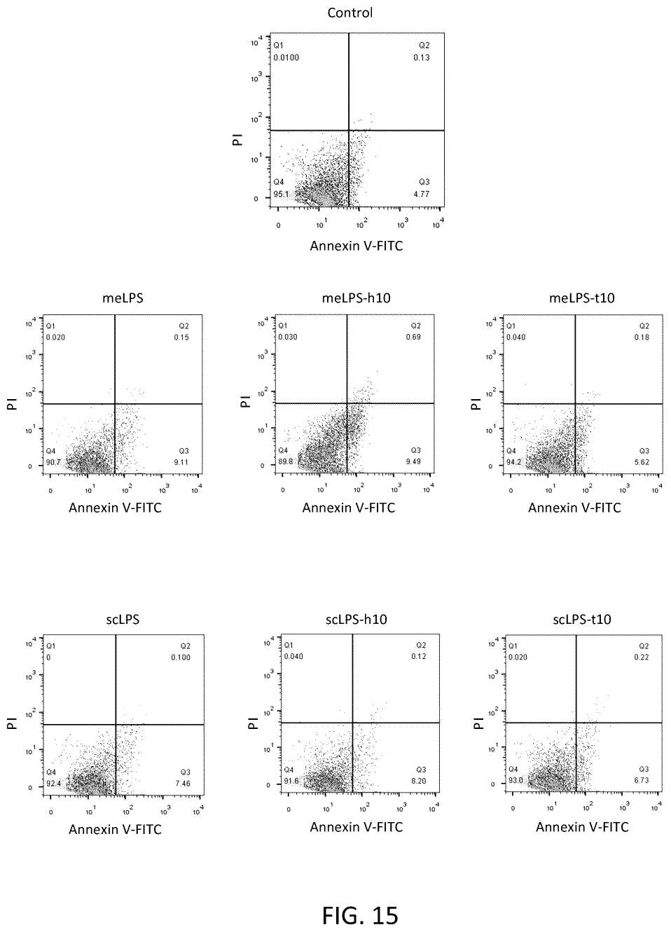

For further investigation of the cytotoxicity of cationic LPSs, they are cultured for 24 hours with JAWS II cells which are double stained with FITC and PI (Propidium Iodide), apoptosis being observed under flow cytometry. Please refer to FIG. 15 which shows results of apoptosis assays of LPSs which are respectively coated with HTCC or TMCC via hydrogen bonds or covalent bonds, as detected by flow cytometry, wherein LPSs of 20 μg/mL are cultured with JAWS II cells for 16 hours, and results are compared with that of LPS without coating HTCC or TMC and control group.

Although cytotoxicity of cationic LPS is detected in previous MTT and LDH assay, in FIG. 15, no significant difference in the ratio of early apoptosis (Q3) to late apoptosis (Q2) is seen among cationic LPS coated with HTCC or TMC, LPS without coating polyssacharide (meLPS and scLPS), and control at a concentration of 20 μg/mL. Similar results are observed in both groups of cationic LPSs coated via hydrogen bonds and covalent bonds. The result implies the cytotoxicity of cationic LPS observed in previous MTT and LDH assay could be a temporary and reversible cell injury, which does not belong to programmed cell death.

### Ability of Cationic LPS to Kill Cancer Cells

As described above, cancer cells show higher uptake selectivity to cationic LPS compared with L929 cells. Cytotoxicity of cationic LPS to immune cells, such as J774A.1 cells and JAWS II cells, is found to be higher than that of neutral or negatively-charged ones. Therefore, a discussion about whether the cytotoxicity of cationic LPS is higher to cancer cells is further provided.

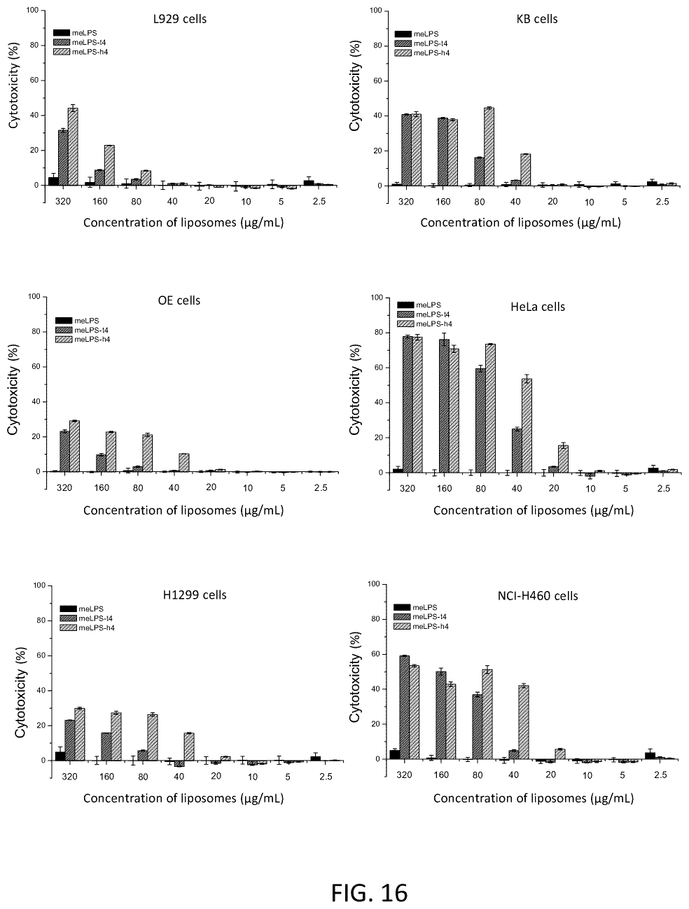

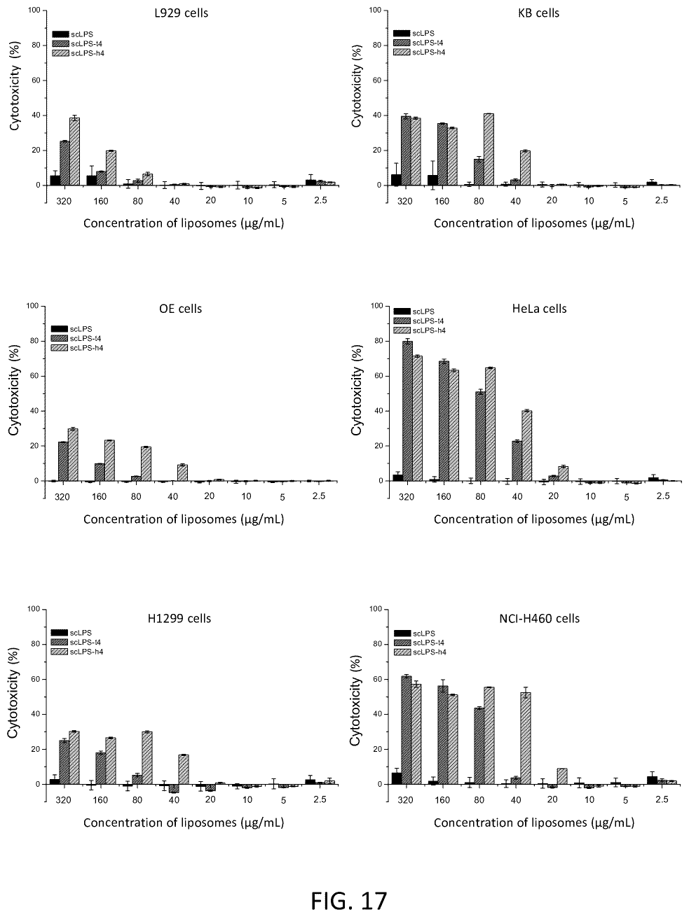

Results of cytotoxicity assay are shown in FIG. 16 and FIG. 17. In FIG. 16, LPSs coated respectively with HTCC or TMC via hydrogen bonds at different concentrations are cultured with L929 cells and cancer cells (KB cells, OE cells, HeLa cells, H1299 cells and NCI-H460 cells) at 37° C. for 24 hours. In FIG. 17, LPSs coated respectively with HTCC or TMC via covalent bonds at different concentrations are cultured with L929 cells and cancer cells (KB cells, OE cells, HeLa cells, H1299 cells and NCI-H460 cells) at 37° C. for 24 hours.

In FIG. 16 and FIG. 17, stronger tumoricidal activity is seen in LPSs coated with HTCC or TMC, via either hydrogen or covalent bonds, compared with uncoated LPSs (meLPS and scLPS), even at a low concentration (<80 μg/mL), with particularly high cytocidal activity against HeLa cells. Furthermore, stronger tumoricidal activity is seen in HTCC-coated LPS compared with TMC-coated ones. The difference is attributed to the high DQ value of HTCC, which gives LPS a greater zeta potential and enhances the uptake of HTCC-coated LPS by cancer cells. In addition, sugar residues of HTCC- or TMC-coated LPS carry a higher percentage of quaternary ammonium groups that results in higher cytotoxicity against cancer cells at a low concentration. It can be deduced that LPS coated with cationic polysaccharides developed in present disclosure is capable of not only targeted cancer cell delivery, but also cancer cell killing. When in synergy with anticancer drugs, increase of therapeutic effect and reduction of required dosage are both achieved, and risk of drug resistance is therefore reduced.

### In Vitro Cytokine Expression of Cationic LPS

Cytokines are proteins that function as chemical messengers and have a specific effect on interactions and communications between cells. Cytokines recruit immune cells to site of inflammation or trauma. Different types of dendritic cells produce different kinds of cytokines. For example, plasmacytoid dendritic cells produce high level of type I interferons, which recruit macrophages to engulf and destroy invading pathogens and other harmful substances. During maturation of dendritic cells, autocrine of cytokines, such as IL-12 and IL-10, affects the induction of Th1 or Th2 immune responses. Besides high levels of antigen presentation and expression of costimulatory molecules, vigorous secretion of IL-12 by mature dendritic cells is reported to be necessary for stimulating innate immunity, which involves production of IFN-γ, an important Th1 inflammatory cytokine. However, release of IL-10 interferes with expression of costimulatory molecules and production of IL-12 and blocks maturation of dendritic cells, thereby keeping dendritic cells from initiating Th1 response. In addition to the above-mentioned cytokines, TNF-α secreted by dendritic cells is also an important inflammatory cytokine in response to invading pathogens or harmful substances. Aside from promoting innate immunity by producing inflammatory cytokines, dendritic cells also initiate adaptive immunity during their maturation to take on what innate immunity fails to eliminate.

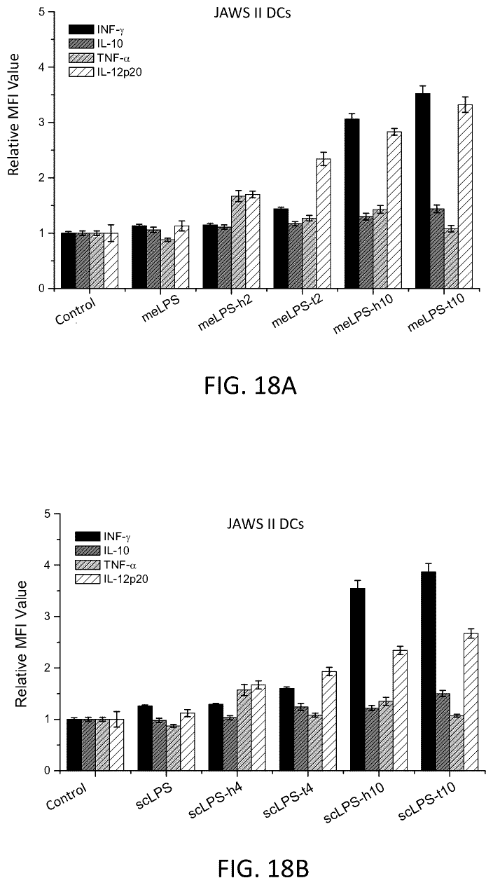

Please refer to FIG. 18A and FIG. 18B. Cytokine levels (IFN-γ, IL-10, TNF-α and IL-12) shown in FIG. 18A are derived from co-culture of JAWS II cells and LPSs which are respectively coated with HTCC or TMC via hydrogen bonds at 37° C. for 24 hours, wherein concentration of LPS is 5 μg/mL. Cytokines levels (IFN-γ, IL-10, TNF-α and IL-12) shown in FIG. 18B are derived from co-culture of JAWS II cells and LPSs which are respectively coated with HTCC or TMC via covalent bonds at 37° C. for 24 hours, wherein concentration of LPS is 5 μg/mL.

According to FIG. 18A and FIG. 18B, IL-12 and IFN-γ levels of co-culture of JAWS II cells and HTCC- or TMC-coated LPS are higher than that of control group, whether LPS is coated via hydrogen bonds or covalent bonds, and the levels increase with the increase of HTCC or TMC concentration. The result indicates that JAWS II dendritic cells secrete more Th1 cytokines when cultured with cationic LPS, in other words, cationic LPS may act as a vaccine adjuvant or carrier and enhance cellular immunity.

Based on the experimental results of cell proliferation, cytotoxicity, and endocytosis, it can be deduced that the cationic LPS developed in the present disclosure which enhances cellular immunity may serve as a carrier or adjuvant for vaccines, especially for the ones that undesirably favor humoral immunity.

Influence of Cationic LPS on Production of Antibody Titers In Vivo after Immunization of Protein Vaccine

Common disadvantages of vaccination include slow induction of immune response, multiple required doses, and short-lived protection.

Taking AVA (Anthracis Vaccine Adsorbed) as an example: six doses of AVA must be vaccinated regularly within 18 months to achieve 100% protection against Bacillus anthracis, and one additional dose is required annually to maintain the effectiveness. Vaccines that require repeated injections are prone to side effects, incomplete immunization, and vaccine refusal.

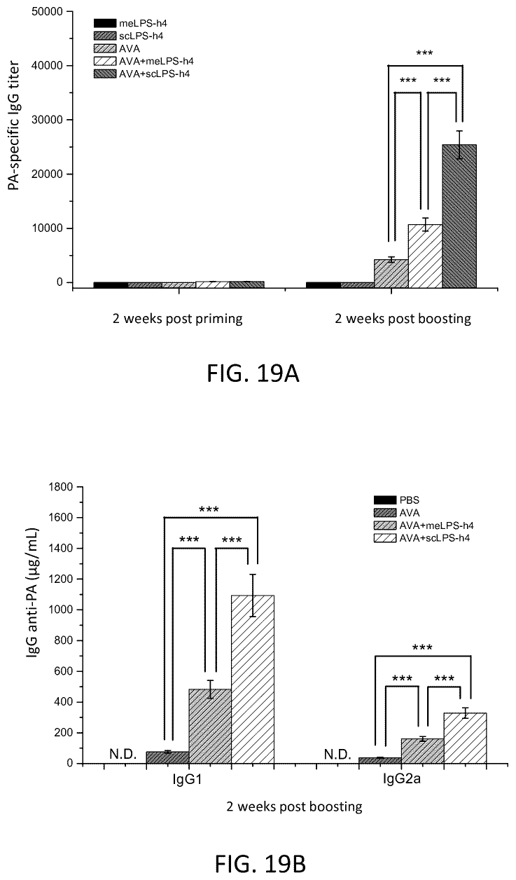

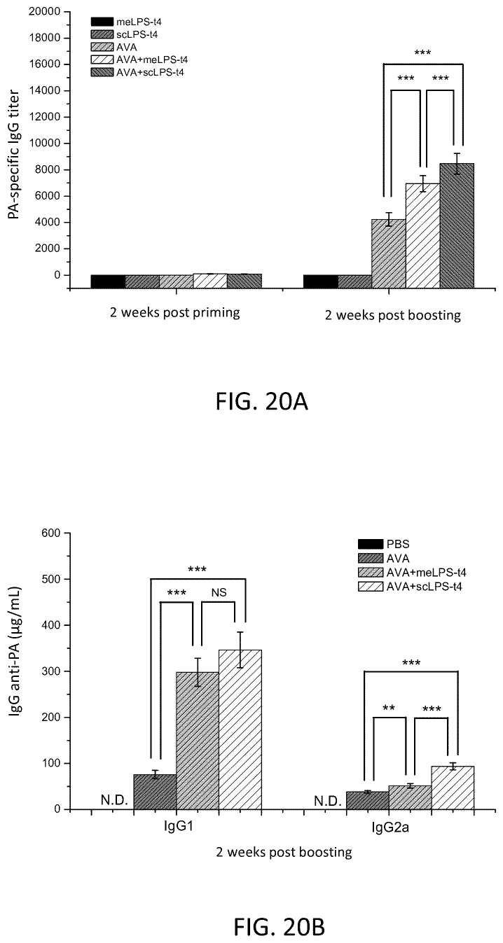

In the present disclosure, whether the developed cationic LPS augments antibody titers induced by protein vaccine is investigated, AVA being the target vaccine. BALB/c mice are vaccinated with a mixture of purchased AVA and the cationic LPS of the present disclosure. Please refer to FIG. 19A, FIG. 19B, FIG. 20A and FIG. 20B. FIG. 19A shows protective antigen (PA)-specific IgG responses in BALB/c mice at two weeks post-priming and two weeks post-boosting in group meLPS-h4, scLPS-h4, AVA, AVA+meLPS-h4 and AVA+scLPS-h4, wherein *p<0.05, **p<0.01, and ***p<0.001. FIG. 19B shows IgG1 and IgG2a subclass distributions in BALB/c mice at two weeks post-boosting in group AVA, AVA+meLPS-h4 and AVA+scLPS-h4, wherein *p<0.05, **p<0.01, and ***p<0.001. FIG. 20A shows protective antigen (PA)-specific IgG responses in BALB/c mice at two weeks post-priming and two weeks post-boosting in group meLPS-t4, scLPS-t4, AVA, AVA+meLPS-t4 and AVA+scLPS-t4, wherein *p<0.05, **p<0.01, and ***p<0.001. FIG. 20B shows IgG1 and IgG2a subclass distribution in BALB/c mice at two weeks post-boosting in group AVA, AVA+meLPS-t4 and AVA+scLPS-t4, wherein *p<0.05, **p<0.01, and ***p<0.001.

According to FIG. 19A, FIG. 19B, FIG. 20A and FIG. 20B, faster and stronger induction of anti-PA IgG is seen in mice vaccinated two times with AVA mixed with HTCC- or TMC-coated cationic LPS compared with the ones vaccinated with only AVA. Mixture of AVA and cationic LPS induces stronger cellular and humoral immunity in mice than sole AVA. The ratio of IgG1 to IgG2 is larger than 1, which means Th2 response, namely humoral immunity, is dominant. LPS coated with cationic polysaccharides via covalent bonds enhances immune response more than the one coated via hydrogen bonds does, and HTCC-coated LPS enhances immune response more than TMC-coated one does.

The cationic LNC developed in the present disclosure could also carry functional compounds. As described above, PEG derivatives of phospholipids may connect to conjugating molecules, for example, NHS, N-maleimide, orthopyridyl disulfide, and vinylsulfone, to bind with peptides, proteins (antibodies) or aptamers. Accordingly, cationic LNC can deliver functional compounds, such as antibodies, with conjugating molecules.

### Application of Cationic LPS in Intranuclear Nucleic Acids Delivery for Translation and Expression Thereof

Nucleic acids, for instance, DNA, RNA, and siRNA, hold great potential for curing diseases, such as cancer, hereditary diseases, and infectious diseases. It is verified above that the cationic LPS developed in the present disclosure provides effective targeted delivery to cancer cells and may serve as vaccine adjuvant. Based on previous discussion, further experiments on the application of cationic LPS in nucleic acid delivery are performed.

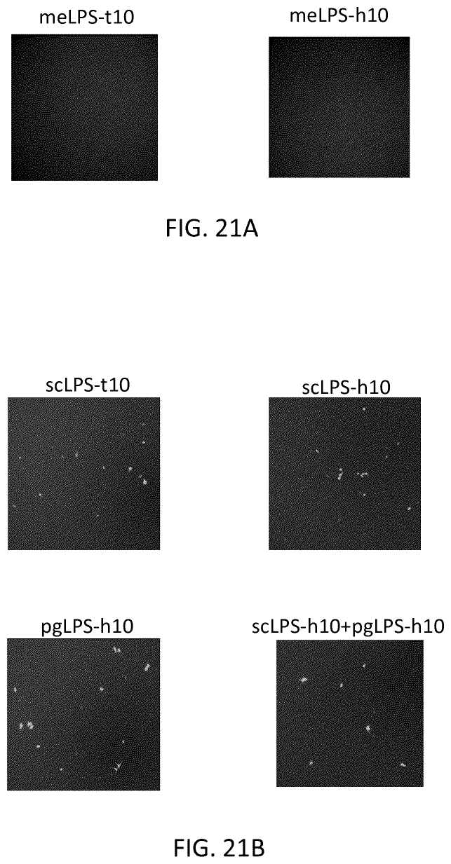

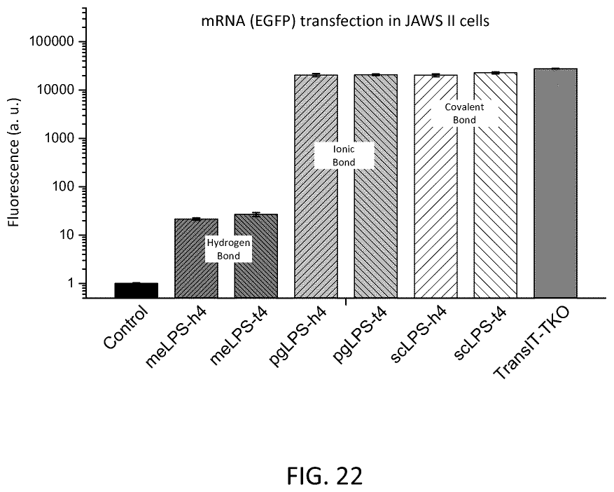

Please refer to FIGS. 21A-21B and FIG. 22. FIG. 21A shows fluorescent images of 293T cells after 48 hours of co-culture with EGFP DNA plasmids that are mixed with meLPS-t10 or meLPS-h10 which are respectively coated with TMC and HTCC via hydrogen bonds in a ratio of 1:20. FIG. 21B shows fluorescent images of 293T cells after 48 hours of co-culture with EGFP plasmids that are mixed with scLPS-t10 or scLPS-h10 which are respectively coated with TMC and HTCC via covalent bonds, pgLPS-h10 which is coated with HTCC via ionic bonds, and pgLPS-h10+scLPS-h10 which is respectively coated with TMC and HTCC via covalent bonds and ionic bonds in a ratio of 1:20. FIG. 22 shows fluoresces generated from EGFP mRNAs which are delivered into JAWS II cells by different kinds of cationic LPSs in a RNA-to-LPS ratio of 1:20.

As shown in FIG. 21A and FIG. 21B, LPS coated with polyelectrolytes via hydrogen bonds (as shown in FIG. 21A) cannot deliver DNA into JAWS II dendritic cells; however, the ones coated with high concentrations of TMC or HTCC via covalent or ionic bonds (as shown in FIG. 21B) are capable of the delivery. A consistent result is also shown in FIG. 22 in which the delivery of EGFP mRNA into JAWS II dendritic cells is detected by flow cytometry. LPS coated with HTCC or TMC via covalent or ionic bonds delivers nucleic acid more effectively than the ones coated via hydrogen bonds.

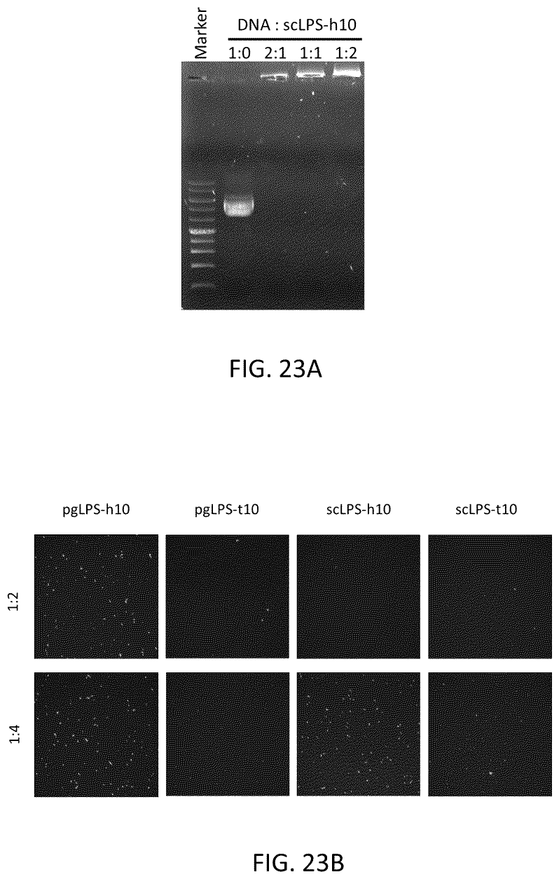

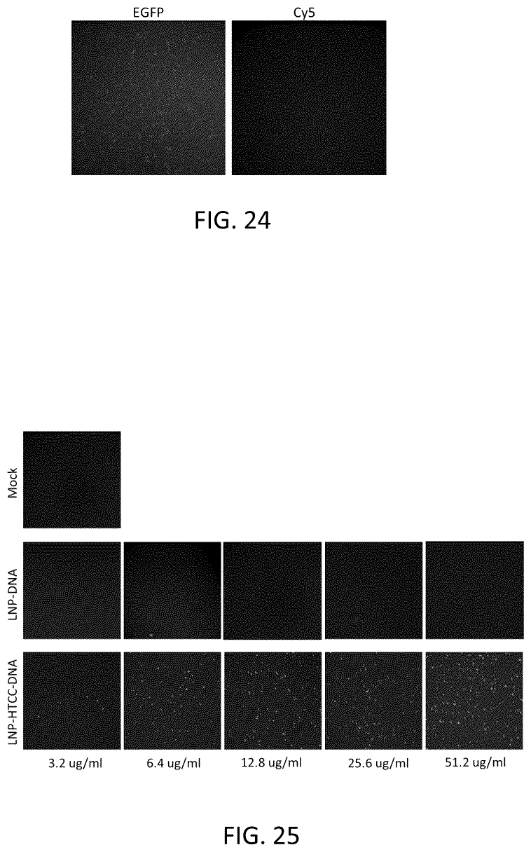

In order to find an optimal mixing ratio of DNA to the cationic LPS, the adsorption of DNA to cationic LPS is tested. Please refer to FIGS. 23A-23B and FIG. 24. FIG. 23A shows adsorptions of DNA to cationic LPSs in different mixing ratios. FIG. 23B shows fluorescent images of 293T cells after 48 hours of co-culture with EGFP plasmid DNAs which are mixed with different cationic LPSs in ratios of 1:2 and 1:4. FIG. 24 shows fluorescent images of 293T cells after co-culture with Cy5-CTP-labeled EGFP mRNA under two kinds of fluorescence after mixing with scLPS-h10 in a ratio of 1:4.

According to the result of gel electrophoresis shown in FIG. 23A, DNA molecules are completely adsorbed to cationic LPS coated with high concentration of HTCC when mixed in a DNA-to-LPS ratio of 1:2 (by weight). As shown in FIG. 23B, fluorescence is optimized when DNA is mixed with the cationic LPS in a ratio from 1:2 to 1:4. As shown in FIG. 24, both indicators of delivery (Cy5) and expression (EGFP) can be observed under a fluorescent microscope. It can be concluded that the cationic LPS effectively delivers nucleic acids, and grants high levels of expression.

### Application of Cationic LNP in Intranuclear Nucleic Acids Delivery for Translation and Expression Thereof

Remarkable development of LNP for mRNA delivery is seen in its application in COVID-19 vaccine. Compared with LPS, LNP, as a carrier, encapsulates and protects mRNA from being degraded by enzymes. However, the naturally low zeta potential of LNP in a neutral environment results in poor adsorption to cells and limits in application. In response, HTCC is coated onto LNP in a single microfluidic step as described above to introduce positive charges thereon for novel cationic LNP formation. The cationic LNP is composed of DODAP (1,2-dioleoyl-3-dimethyl-ammoniumpropane), soy-PC, cholesterol, and DSPE-PEG5k-Me in a molar ratio of 50:10:38.5:1.5. The aqueous phase is citrate buffer (pH 4.0), the organic phase is ethanol, the concentration of HTCC is 10 mg/mL, TFR is 12 mL/min, FRR of aqueous to organic phase is 3:1, and final concentration of EGFP DNA plasmid is 125 μg/mL. The obtained LNP-DNA is neutral with a particle size of 82 nm and a zeta potential of +0.6 mV after dialysis, and the obtained cationic LPN-HTCC-DNA after dialysis has a particle size of 52 nm and a zeta potential of +7.2 mV.

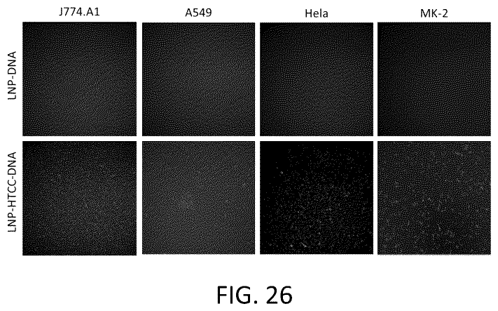

Please refer to FIG. 25 and FIG. 26. FIG. 25 shows fluorescent images of 293T cells after 48 hours of co-culture with the neutral LNP with DNA embedded therein (LNP-DNA) and the cationic LNP with DNA embedded therein (LNP-HTCC-DNA), which are respectively prepared in the single microfluidic step at different concentrations. FIG. 26 shows fluorescent images of various cells cultured with the neutral LNP with DNA embedded therein (LNP-DNA) and the cationic LNP with DNA embedded therein (LNP-HTCC-DNA).

As shown in FIG. 25, LNP-HTCC-DNA successfully transfects EGFP plasmid DNA into the nucleus of co-cultured cells for expression, wherein dose-dependent increase in fluorescence is observed, while no fluorescence signal is detected in the case of neutral LNP-DNA. As shown in FIG. 26, the delivery ability of LNP-HTCC-DNA is greater than that of the conventional neutral LNP in multiple kinds of cells.

In conclusion, the present disclosure provides single-step microfluidic preparation of cationic LNC which is coated with polyelectrolytes (HTCC or TMC) on the surface thereof via different chemical bonds, where the preparation is simplified, the cost is reduced, and the controller parameters are shared between laboratory and factory microfluidic devices which allow efficient scale-up. The prepared cationic LNC has been proven to be capable of increasing the efficiency of endocytosis, by J774A.1 and JAWS II immune cells, L929 cells, and cancer cells, such as KB cells, HeLa cells, NCI-H460 cells, H1299 cells, OE cells, etc., and effectively delivering drugs to targeted cancer cells. Moreover, the cationic LNC itself possesses tumoricidal activity which is positively correlated with the concentration of the coated cationic polysaccharides, whereby, when combined with anticancer drugs, therapeutic effect is synergized and required doses are reduced. Likewise, in vaccination, mixture of the cationic LNC with vaccines enhances induction of antibody titers, thereby reducing required doses of vaccination to achieve prompt and long-lived protection. The cationic LNC also serves as a carrier through conjugating molecules for effective functional compound delivery. The LPS coated with cationic polysaccharides via ionic bonds and/or covalent bonds effectively transfects nucleic acids into nuclei after simple mixture with DNA or mRNA. The cationic LNP with DNA embedded therein is similarly effective. In sum, the cationic LNC developed in present disclosure serves as an excellent delivery system for various substances, such as small molecular compounds, proteins, and nucleic acids, of which the preparation is simple and easy to scale up, whereby the present disclosure possesses competitiveness and practicability in related industries.

While the disclosure has been described in terms of what is presently considered to be the most practical and preferred embodiments, it is to be understood that the disclosure needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A method for preparing a cationic lipid-based nanocarrier, comprising:
providing an organic solvent and an aqueous solution;
adding compositions for forming a lipid-based nanocarrier and at least one kind of cationic polysaccharides respectively into the organic solvent and the aqueous solution according to respective solubility thereof, and
flowing the organic solvent and the aqueous solution through a microfluidic device under the control of a micromixer, so as to mix the organic solvent and the aqueous solution in the microfluidic device, thereby obtaining the cationic lipid-based nanocarrier in a single-step process.

2. The method for preparing the cationic lipid-based nanocarrier according to claim 1, wherein the cationic lipid-based nanocarrier is formed by coating the at least one kind of cationic polysaccharides on an outer surface of the lipid-based nanocarrier via hydrogen bonds, ionic bonds, covalent bonds or a combination thereof.

3. The method for preparing the cationic lipid-based nanocarrier according to claim 1, wherein the at least one kind of cationic polysaccharides comprises at least one selected from the group consisting of N-[(2-hydroxy-3-trimethylammonium)propyl]chitosan chloride (HTCC) and N-trimethylchitosan (TMC), and the at least one kind of cationic polysaccharides has a molecular weight of 5-1000 kDa, a degree of deacetylation (DD) of 50-99%, and a degree of quaternization (DQ) of 20-90%.

4. The method for preparing the cationic lipid-based nanocarrier according to claim 1, wherein a flow rate ratio (FRR) of the aqueous solution and the organic solvent is controlled by the micromixer at 10:1 to 1:10, a total flow rate (TFR) is controlled by the micromixer at 1 to 40 mL/min, and a total lipid concentration (TLC) is of 1 to 100 mg/mL.

5. The method for preparing the cationic lipid-based nanocarrier according to claim 1, wherein the compositions for forming the lipid-based nanocarrier comprise phospholipid and at least one selected from the group consisting of cholesterol, polyethylene glycol (PEG) derivative of phospholipid, and ionizable lipid or cationic lipid.

6. The method for preparing the cationic lipid-based nanocarrier according to claim 5, wherein the phospholipid is phosphatidic acid (PA) type phospholipid comprising one glycerol molecule connected to two saturated or unsaturated fatty acids with C16-C22 chain and one phosphate molecule.

7. The method for preparing the cationic lipid-based nanocarrier according to claim 6, wherein the PA-type phospholipid is further connected with a polar molecule to form at least one type of phospholipid selected from the group consisting of phosphatidylcholine (PC) type phospholipid, phosphatidylethanolamine (PE) type phospholipid, phosphatidylserine (PS) type phospholipid, phosphatidylinositol (PI) type phospholipid, and phosphatidylglycerol (PG) type phospholipid.

8. The method for preparing the cationic lipid-based nanocarrier according to claim 5, wherein the polyethylene glycol (PEG) derivative of phospholipid comprises a phosphatidylethanolamine (PE) type phospholipid connected with a polyethylene glycol having a molecular weight of 500-1000000 Da.

9. The method for preparing the cationic lipid-based nanocarrier according to claim 5, wherein one end of the polyethylene glycol (PEG) derivative of phospholipid is connected with one selected from the group consisting of a folic acid, a biotin, mannose, a galactose, and a cRGD peptide, or is connected with a conjugating molecule selected from the group consisting of N-Hydroxysuccinimide (NHS), N-maleimide, orthopyridyl disulfide, and vinylsulfone.

10. The method for preparing the cationic lipid-based nanocarrier according to claim 5, wherein the ionizable lipid or cationic lipid comprises a C16-C100 saturated or unsaturated fatty acid chain with a primary amine, a secondary amine, a tertiary amine or a quaternary amine.

11. The method for preparing the cationic lipid-based nanocarrier according to claim 1, further comprising a step of adding a bioactive or functional substance to be delivered by the cationic lipid-based nanocarrier into the organic solvent or the aqueous solution according to solubility thereof.

12. The method for preparing the cationic lipid-based nanocarrier according to claim 11, wherein the bioactive or functional substance comprises at least one selected from the group consisting of small molecular drug, peptide, protein, nucleic acid, fluorescent molecule, photosensitive reagent, nanometal and quantum dots.

13. The method for preparing the cationic lipid-based nanocarrier according to claim 11, wherein the organic solvent and the aqueous solution have dissolved therein 10-100 mole % of soy-PC, egg-PC, PC-type phospholipid, PE-type phospholipid, PS-type phospholipid, PI-type phospholipid, PG-type phospholipid, PA-type phospholipid or a combination thereof, 0-50 mole % of cholesterol, 0-50 mole % of PEG derivative of PE-type phospholipid, 0-20 mole % of bioactive or functional substances, and 0.1-40 mg/mL of HTCC, TMC or a combination thereof, and wherein the organic solvent comprises one selected from the group consisting of alcohol with less than or equal to 4 carbons, Dimethyl sulfoxide (DMSO), Dimethylformamide (DMF) or a mixed solution thereof, and the aqueous solution comprises water or a buffer solution.

14. The method for preparing the cationic lipid-based nanocarrier according to claim 11, wherein the organic solvent and the aqueous solution have dissolved therein 10-100 mole % of soy-PC, egg-PC, PC-type phospholipid, PE-type phospholipid, PS-type phospholipid, PI-type phospholipid, PG-type phospholipid, PA-type phospholipid or a combination thereof, 0-50 mole % of cholesterol, 0-50 mole % of PEG derivative of PE-type phospholipid, 1-60 mole % of ionizable lipid or cationic lipid, 1-20 mole % of nucleic acid, and 0.1-40 mg/mL of HTCC, TMC or a combination thereof, and wherein the organic solvent comprises one selected from the group consisting of alcohol with less than or equal to 4 carbons, Dimethyl sulfoxide (DMSO), Dimethylformamide (DMF) or a mixed solution thereof, and the aqueous solution comprises water or a buffer solution.

15. The method for preparing the cationic lipid-based nanocarrier according to claim 1, wherein the cationic lipid-based nanocarrier has a particle size ranged between 10 nm and 500 nm and a zeta potential ranged between +2 mV and +60 mV.

16. The method for preparing the cationic lipid-based nanocarrier according to claim 1, wherein the lipid-based nanocarrier comprises at least one of a liposome and a lipid nanoparticle.

17. A cationic lipid-based nanocarrier prepared by the method according to claim 1.

18. The cationic lipid-based nanocarrier according to claim 17, wherein the cationic lipid-based nanocarrier is used as one selected from the group consisting of a protein carrier, a nucleic acid carrier, a small molecular drug carrier and a vaccine adjuvant.
